(19) United States
(12) Patent Application Publication
(10) Pub. No.: US 2025/0057414 A1
(43) Pub. Date: Feb. 20, 2025

(54) SYSTEMS, DEVICES, AND METHODS FOR IMAGING A SUBJECT’S RETINA AND ANALYZING OF RETINAL IMAGES

(71) Applicant: C. Light Technologies, Inc., Medford, MA (US)

(72) Inventors: Christy K. Sheehy, Medford, MA (US); Mark Mackanos, San Jose, CA (US); Jason Karp, Portland, MA (US); Daniel Gray, Portland, MA (US); Scott Liddle, Cary, NC (US); Nathan Luck, Cary, NC (US); Joe Xing, San Jose, CA (US); Calen Walshe, Seattle, WA (US); Andrew Norton, Santa Barbara, CA (US); Jacqueline Theis, Richmond, VA (US)

(73) Assignee: C. Light Technologies, Inc., Medford, MA (US)

(21) Appl. No.: 18/723,333

(22) PCT Filed: Dec. 22, 2022
(86) PCT No.: PCT/US2022/053853

§ 371 Date: Jun. 21, 2024

Related U.S. Application Data
(60) Provisional application No. 63/340,898, filed on May 11, 2022.
(60) Provisional application No. 63/297,917, filed on Jan. 10, 2022.
(60) Provisional application No. 63/297,920, filed on Jan. 10, 2022.
(60) Provisional application No. 63/297,932, filed on Jan. 10, 2022.
(60) Provisional application No. 63/293,655, filed on Jan. 8, 2022.
(60) Provisional application No. 63/293,656, filed on Dec. 23, 2021.
(60) Provisional application No. 63/293,657, filed on Dec. 23, 2021.
(60) Provisional application No. 63/293,658, filed on Dec. 23, 2021.

(51) Int. Cl. A61B 3/10; A61B 3/12; A61B 3/14

(52) U.S. Cl. CPC A61B 3/1025; A61B 3/12; A61B 3/14

(57) ABSTRACT

A monocular and/or a binocular system may be used to generate one or more images, or videos, of a subject's retina by scanning in one or two directions in one or two dimensions to, for example, measure and/or report fixation, smooth pursuit, and/or saccadic responses of the subject's eye. The scanning and/or image generation process may be optimized to correct for distortions in the subject's eye and/or improve SNR among one or a plurality of retinal images. In some cases, the retinal images may be used to train a retinal feature detection model and/or algorithm that may be used to make predictions and/or inferences regarding a visual pattern showing features of subsequently received retinal images. These models and/or predicted patterns present within the models may be used by the retinal feature detection model and/or algorithm to monitor features of the retina and, in some instances, track voluntary and/or involuntary eye motion.

### RELATED APPLICATIONS

The instant patent application is an INTERNATIONAL patent application that claims priority to U.S. Provisional Patent Application No. 63/293,656, filed 23 Dec. 2021, and entitled “SYSTEM AND METHOD FOR GENERATING A COMPOSITE RETINAL IMAGE;” U.S. Provisional Patent Application No. 63/293,657, filed 23 Dec. 2021, and entitled “A DUAL-DEFOCUS CORRECTION SYSTEM FOR A SCANNING LASER OPHTHALMOSCOPE AND METHODS OF USE THEREOF;” U.S. Provisional Patent Application No. 63/293,658, filed 23 Dec. 2021, and entitled A FIXATION TARGET DISPLAY FOR A SCANNING LASER OPHTHALMOSCOPY SYSTEM AND METHODS OF USE THEREOF;” U.S. Provisional Patent Application No. 63/293,655, filed 8 Jan. 2022, and entitled “BI-DIRECTIONALLY SCANNING LASER OPHTHALMOSCOPY SYSTEM AND METHODS OF USE THEREOF;” U.S. Provisional Patent Application No. 63/297,917, filed 20 Jan. 2022 and entitled “A SIGNAL QUALITY EVALUATION SYSTEM FOR USE WITH IMAGES CAPTURED BY A SCANNING LASER OPHTHALMOSCOPY SYSTEM AND METHODS OF USE THEREOF;” U.S. Provisional Patent Application No. 63/297,920, filed 20 Jan. 2022, and entitled “SYSTEMS AND PROCESSES FOR TRAINING A RETINAL FEATURE DETECTION MODEL AND METHODS OF USE THEREOF;” U.S. Provisional Patent Application No. 63/297,932 filed 20 Jan. 2022, and entitled “SYSTEMS AND METHODS FOR DETECTING RETINAL FEATURES AND/OR ANALYZING CHARACTERISTICS OF RETINAL MOTION OVER TIME;” and U.S. Provisional Patent Application No. 63/340,898, filed 11 May 2022, and entitled “CHIN AND HEAD REST SYSTEMS AND DEVICES FOR USE WHEN SCANNING A SUBJECT'S RETINA AND METHODS OF USE THEREOF” all of which are incorporated herein in their entirety.

### FIELD

The present invention pertains to chin and head rest systems and devices for use when scanning a subject's retina using, for example, a scanning laser ophthalmoscopy (SLO) system, a bi-directional SLO system, a tracking scanning laser ophthalmoscopy system (TSLO) system, a bi-directional TSLO system, an autorefractor, autokeratometer, corneal pachymeter, slit lamp, and/or an optical coherence tomography (OCT) system, and methods of use thereof. The present invention also pertains to a signal quality indicator and/or evaluation system for use with signals and/or images (e.g., retinal images) captured using an SLO and/or a TSLO and methods of use thereof. The present invention further pertains to systems and processes for eye tracking and more particularly to systems and methods for automatically detecting and/or analyzing retinal characteristics and/or retinal movement over time that may include generation of a retinal feature detection model via, for example, machine learning and/or use of a deep neural network.

### BACKGROUND

With the advent of eye-tracking using high-resolution retinal imaging systems, such as scanning laser ophthalmoscopy, precise real-time eye tracking at sub-micron resolution is now possible. However, the constraints that come from real-time image signal quality can result in a relatively high failure rate of motion extraction for applications to successfully track eye motion information using strip-based image registration methods.

### SUMMARY

The device described here uses, for example, a low power laser beam in a scanning laser ophthalmoscopy (SLO) system to raster or line scan in one or two dimensions over an eye's retina is herein disclosed. The reflected (or returned) light is detected and used to generate a digital image and/or series of digital images (e.g., a video of the retina with, for example, a computer or electronic imaging device that may utilize retinal eye tracking to measure and report, for example, movement of the retina, and/or a movement indicative of a saccadic, fixation, and/or smooth pursuit response. Additionally, or alternatively, the system may be configured for recording, viewing, measuring, and/or analyzing temporal characteristics of, for example, saccadic, smooth pursuit, and/or fixation responses when a subject is viewing a static or dynamic visual stimuli and identifying metrics and stability of these movements. The SLO system may be monocular or binocular.

Following image generation, the images may be analyzed to measure eye/retinal motion, and in particular may be analyzed to measure fixational retinal motion and/or measure a number, and/or characteristics, of saccades and/or microsaccades, smooth pursuit, and/or drift. When fixational eye motion is being measured, this data may be gathered when a subject fixates on a target and series of images are captured by the SLO device. These images may be analyzed to measure, for example, metrics quantifying the fixational eye movement such as translational retinal movement over time, drift, and characteristics of saccades and/or microsaccades. Additionally, these images may be analyzed to measure smooth pursuit, blink rate, and spontaneous venous pulsation of the optic nerve.

In addition, systems, devices, and methods for imaging a retina and analyzing these images in order to, for example, determine characteristics of retinal/eye motion that incorporates a scanning laser ophthalmoscope (SLO) may be used to capture retinal images used to train a retinal feature detection model and/or algorithm using, for example, machine learning and/or a deep neural network. The retinal feature detection model and/or algorithm may then be used to make predictions and/or inferences regarding a visual pattern showing features (e.g., blood vessels and/or a capillary network) of subsequently received retinal images, which may model the retinal images. These models and/or predicted patterns present within the models may be used by the retinal feature detection model and/or algorithm to monitor features of the retinal and, in some instances, track voluntary and/or involuntary eye motion over time.

In some embodiments, detection path data (e.g., a light signal reflected from a subject's eye and/or a digital signal generated responsively to a light signal reflected from a subject's eye) may then be analyzed to determine of it is of sufficient strength and/or quality for further analysis to, for example, determine retinal motion over time. An indicator of sufficient strength and/or quality is signal-to-noise (SNR) ratio, or other metric related to signal strength, above a threshold value. When the signal quality for an image and/or set of images is too low and/or not able to be resolved into an image with distinguishable features, the signal representing the image and/or set of images may be rejected so that, for example, another measurement/imaging of a subject's eye(s) may be taken and/or a set of images may be filtered to remove frames that do not have a high enough resolution (e.g., signal-to-noise ratio) to be used for further analysis. When the signal quality is sufficient and/or strong enough, one or more retinal images may be rendered using the signal and then analyzed to measure, for example, eye/retinal motion, and in particular may be analyzed to measure fixational retinal motion and/or measure a number, and/or characteristics, of saccades and/or microsaccades, smooth pursuit, bink rate, and/or drift. When fixational eye motion is being measured, this data may be gathered when a subject fixates on a target, or a series of targets and a sequential series of retinal images are captured by the SLO device. These images may be analyzed to measure, for example, metrics quantifying the fixational, saccadic, and/or smooth pursuit eye movement such as translational retinal movement over time, drift, and characteristics of saccades and/or microsaccades. Additionally, or alternatively, these images may be analyzed to determine differences between anatomical features shown in the images.

When the images are analyzed to measure a number, and/or characteristics, of saccades, a subject may be provided with two or more targets (e.g., crosshairs) to alternately focus on. When in use, the subject may move his or her eyes voluntarily back and forth between the targets (in the same direction of the target (saccade) or in the equal but opposite direction (antisaccade)) and analysis of images of the subject's retina (taken using the system disclosed herein) while voluntarily moving his or her eyes back and forth may allow for the quantification of both horizontal and vertical saccades. In many instances, the two targets may be separated by, for example, a visual angle of 0.5-8 degrees along the same horizontal and/or vertical axis. Additionally, a single moving target may be presented to elicit a voluntary smooth pursuit movement that is produced when following the moving target as it moves on the screen.

Some embodiments of the present invention may include a scanning laser ophthalmoscope (SLO) imaging system with monocular and/or binocular imaging optics configured to image a retina of a subject's right and/or left eye of a subject. In some embodiments, the system may include a camera arranged and configured to enable an operator to view the subject's eye and/or pupil and/or aide the operator in aligning the subject's eye and/or pupil with the imaging system. The monocular imaging optics may include a scanning radiation source (e.g., a super luminescent diode) may be arranged and configured to emit a beam of scanning radiation for imaging the subject's retina toward a fiber collimator. The fiber collimator may be arranged and configured to receive the beam of scanning radiation from the scanning radiation source, collimate the beam of scanning radiation, thereby generating a collimated beam of scanning radiation, and direct the collimated beam of scanning radiation to a first beam splitter. The first beam splitter may be may be arranged and configured to direct the collimated beam of scanning radiation to a scan path defocus correction assembly may be arranged and configured to receive the collimated beam of scanning radiation, apply a defocus and/or spherical equivalent correction of a subject's eye or eyes to the collimated beam of scanning radiation, thereby generating a corrected beam of scanning radiation, and direct the corrected beam of scanning radiation through an iris toward a first mirror. In some embodiments, the scan path defocus correction assembly may be opto-mechanically controlled. Additionally, or alternatively, the scan path defocus correction assembly may include two or more lenses. A degree, or feature, of the defocus and/or spherical equivalent correction applied to the collimated beam of scanning radiation may be responsive to imperfections of the subject's eye and/or lens so that, for example, these imperfections to not impact the resolution and/or accuracy of the images of the subject's retina. Additionally, or alternatively, a degree, amount, and/or feature of the defocus or spherical equivalent correction applied to the collimated beam of scanning radiation by the scan path defocus correction assembly may be responsive to an analysis of retinal image quality and may be applied to improve (e.g., reduce blurriness, resolve imaged retinal features with better clarity, etc.) retinal image quality. At times, the defocus or spherical equivalent correction of a subject's eye or eyes applied to the collimated beam of scanning radiation by the scan path defocus correction assembly may be within a range of −12 diopters to +12 diopters.

The first mirror may be arranged and configured to direct the corrected beam of scanning radiation to a fast-scanner optical element that may be arranged and configured to receive the corrected beam of scanning radiation and direct the corrected beam of scanning radiation toward a second scanning mirror that may be arranged and configured to receive the corrected beam of scanning radiation and direct the corrected beam of scanning radiation toward an optical element. In some embodiments, wherein the fast-scanning optical element may be a mirror Additionally, or alternatively, wherein the fast-scanning optical element may be arranged and configured to steer the corrected beam of scanning radiation toward the slow-scanning optical element along a first scanning dimension (e.g., along the X-axis). In some embodiments, the slow-scanning optical element may be arranged and configured to direct the corrected beam of scanning radiation toward the optical element along a second scanning dimension (e.g., along the Y-axis).

The optical element may be arranged and configured to receive the corrected beam of scanning radiation and direct the corrected beam of scanning radiation to a second beam splitter that may be arranged and configured to direct the corrected beam of scanning radiation toward a relay element that may be arranged and configured direct the corrected beam of scanning radiation onto a subject's pupil and/or retina, thereby imaging the retina. The scanning radiation may then reflect off of the subject's retina and be directed through the imaging optics described above however, when the reflected scanning radiation reaches the first beam splitter, the first beam splitter may direct the reflected scanning radiation to a detector assembly along a detection path. The detector assembly may include a focusing lens that may be arranged and configured to receive scanning radiation reflected from the subject's retina via the first beam splitter and focus the radiation reflected from the subject's retina onto an imaging system that may be arranged and configured to receive scanning radiation reflected from the subject's retina via the first beam splitter and communicate an indication of the scanning radiation reflected from the subject's retina to an external computing device, such as a processor or cloud computing environment. In some embodiments, the system may further include an acousto-optic modulator (AOM) configured to generate a fixation target (e.g., an image or series of images) for the subject. The fixation target may be configured to guide a focal position for the subject and/or facilitate voluntary and/or fixational motion of the subject's eye that, in some embodiments, may yield the scanning of predictable fields of view of the subject's retina.

In some embodiments, a first image of a first field of view of a subject's retina and a second image of the retina of a second field of view of the retina may be received. A portion of the retina shown along a first (e.g., right side) edge of the first image and a first (e.g., left side) edge of the second image may be the same as may happen when, for example, a portion of the first and second fields of view overlap. The first edge of the first image and the first edge of the second image may be aligned so that the first edge of the first image overlaps the first edge of the second image, thereby generating a composite retinal image that shows the first field of view and the second field of view. Optionally, generation of the composite retinal image may include removal of any duplicate areas and/or filling in gaps (e.g., empty space, or blurry areas) that may be present between the first and second images. Gaps between the first and second composite images may be made by, for example, analyzing features of the first and second images to determine how a feature that straddles both the first and second image may appear in the empty space between the first and second images.

Optionally, a third image of a third field of view of the retina may be received and a portion of the retina shown along a first (e.g., upper) edge of the third image and a second (e.g., lower) edge of the first image is the same. The first edge of the third image and the second edge of the first image may be aligned so that the first edge of the third image overlaps the second edge of the first image, thereby generating a second composite retinal image that shows the first field of view, the second field of view, and the third field of view. At times, a fourth image of a fourth field of view of the retina may be received and a portion of the retina shown along a first (e.g., upper) edge of the fourth image and a second (e.g., lower) edge of the second image is the same. The first edge of the fourth image and the second edge of the second image may be aligned so that the first edge of the fourth image overlaps the second edge of the second image, thereby generating a third composite retinal image that shows the first, second, third, and fourth fields of view. Optionally, when forming the third composite retinal image, a second (e.g., left) edge of the fourth image and a second (e.g., right) edge of the third image so that the second edge of the fourth image overlaps the second edge of the third image.

At times, the composite retinal image may be used as a reference frame against which other images of the subject's retina may be analyzed. For example, a fifth image of the subject's retina may be received and compared with the first, second, or third composite retinal image to, for example, determine a position of the fifth image within at least one of the first field of view, the second field of view, and the third field of view, and the fourth field of view based on a comparison of the fifth image with the composite image. Optionally, a sixth image of the retina may be received and compared with the composite image to determine a position of the sixth image within at least one of the first field of view, the second field of view, the third field of view, and the fourth field of view based on the comparison of the sixth image with the composite image. Then, a change in position between the fifth image and the sixth image may be determined and a characteristic of retinal motion between the fifth and sixth images may be determined and provided to an operator and/or the subject. The characteristic may be, for example, a velocity of retinal motion, a direction of retinal motion, a magnitude of retinal motion, a speed of retinal motion, a magnitude of drift, and a velocity of drift.

In some embodiments, a set of detection path signals may be received from an optical array like the optical array(s) disclosed herein. The detection path signals may correspond to a plurality of scans of a subject's retina taken over a time interval, The set of detection path signals may be processed to deconvolve, filter, reduce noise, and/or otherwise generate or render a plurality of images of the subject's retina taken over a period (e.g., 3-300 seconds), or interval, of time. In some instances, the detection path signals and/or retinal images generated therefrom may be collected as the subject voluntarily moves his or her retina over the time interval. Additionally, or alternatively, the detection path signals and/or retinal images generated therefrom may be collected as the subject fixates his or her retina on a plurality of fixational targets arranged in different positions so that the imaged field of view changes over the time interval. Additionally, or alternatively, the detection path signals and/or retinal images generated therefrom may be collected as the subject fixates his or her retina on a fixational target over the time interval. In these embodiments, retinal images that may be used to deduce characteristics of the subject's fixational eye motion may be captured

Image quality (e.g., a signal to noise ratio, resolution, luminance level, contrast, etc.) for each retinal image of the plurality of images of the subject's retina may be determined and, if image quality for a particular retinal image of the plurality of images falls below a threshold value (e.g., too noisy, poor contrast ratio, too dark to identify or resolve retinal features within the retinal image and/or too blurry), the particular retinal image may be removed from the plurality of retinal images, thereby generating an edited set of images of the subject's retina that may include, for example, only retinal images of a sufficient quality to enable further analysis and/or viewing of the subject's retina. In some cases, the retinal images that do not have sufficient image quality correspond to periods of time during the scanning interval in which the subject was blinking. Removing poor quality images from the set of retinal images enables faster and more accurate processing and analysis of the edited set of images of the subject's retina that the original set of images of the subject's retina.

In some embodiments, determining the image quality includes performing a frequency spectrum analysis on each retinal image. Additionally, or alternatively, determining the image quality may include determining a relationship between frequency and intensity for each retinal image of the plurality of images of the subject's retina.

In some embodiments, a preferred luminance level range and/or contrast level for retinal images may be received and it may be determined whether a luminance and/or contrast level for each retinal image of the edited set of images of the subject's retina falls within the preferred luminance and/or contrast level range and, if not, the luminance and/or contrast level for each of the retinal images included in the edited set of images of the subject's retina that does not fall within the preferred luminance and/or contrast level range for retinal images may be adjusted so that it falls within the preferred range.

In some embodiments, each retinal image included in the edited set of images of the subject's retina may be analyzed to determine differences therebetween and an indication of any determined difference(s) may be provided to an operator. For example, each retinal image included in the edited set of images of the subject's retina may be analyzed to determine a characteristic thereof and a determined characteristic of at least two retinal images may be compared to one another. Then, an indication of the comparison may be provided to the operator. In some cases, the determined characteristic may be a position of a feature shown in the at least two retinal images and a speed, direction, and/or velocity of retinal motion over a time interval between the capture of the at least two images may be determined using the position of the feature shown in the at least two retinal images. Exemplary determined characteristics include, but are not limited to, a direction of retinal motion, a magnitude of retinal motion, a speed of retinal motion, a magnitude of drift, and a velocity of drift.

In some embodiments, a set of retinal images may be received and analyzed to automatically detect a feature (e.g., a blood vessel, fovea, tumbling E, etc.) of each retinal image in the set of retinal images and determine a characteristic (e.g., size, shape, orientation, position, etc.) of the feature of each retinal image in the set of retinal images. On some occasions, the set of retinal images may be part of a series of images taken over a time interval such as a video

Then, a set of visual patterns that, in some cases, may approximate and/or resemble the detected feature may be generated using, for example, each of the automatically detected features, wherein each visual pattern of the set of visual patterns corresponds to a respective retinal image of set of retinal images. In some embodiments, generating a visual pattern of the set of visual patterns includes generating a set of images that includes the respective visual patterns.

In some embodiments, each visual pattern of the set of visual patterns may be analyzed to determine one or more characteristics of the set of visual patterns. In some cases, the characteristic may be a time-based characteristic such as velocity or speed. Additionally, or alternatively, the visual patterns may be analyzed to determine a characteristic of retinal motion.

### BRIEF DESCRIPTION OF THE DRAWINGS

The present invention is illustrated by way of example, and not limitation, in the figures of the accompanying drawings in which:

FIG. 1 is a block diagram illustrating an exemplary SLO retinal imaging and analysis system, consistent with some embodiments of the present invention.

FIG. 2A is a schematic diagram of a rear perspective view of a temple pad, in accordance with some embodiments of the present invention.

FIG. 2B is a schematic diagram of a front perspective view of a temple arm, in accordance with some embodiments of the present invention.

FIG. 3A is a schematic diagram of a front plan view of a torque hinge, in accordance with some embodiments of the present invention.

FIG. 3B is a schematic diagram of a front perspective view of the torque hinge of FIG. 3A, in accordance with some embodiments of the present invention.

FIG. 4A is a schematic diagram of a rear view of a temple pad assembly, in accordance with some embodiments of the present invention.

FIG. 4B is a schematic diagram of a side view of the temple pad assembly of FIG. 4A, in accordance with some embodiments of the present invention.

FIG. 4C is a schematic diagram of a rear perspective view of the temple pad assembly of FIG. 4A, in accordance with some embodiments of the present invention.

FIG. 4D is a schematic diagram of a bottom view of the temple pad assembly of FIG. 4A, in accordance with some embodiments of the present invention.

FIG. 4E is a schematic diagram of the temple pad assembly of FIG. 4A in a first, retracted, position transitioning from a retracted to an open state, in accordance with some embodiments of the present invention.

FIG. 4F is a schematic diagram of the temple pad assembly of FIG. 4A in a second position as the temple pad assembly transitions from the retracted, position of FIG. 4E to an open state, in accordance with some embodiments of the present invention.

FIG. 4G is a schematic diagram of the temple pad assembly of FIG. 4A in a third, open position, in accordance with some embodiments of the present invention.

FIG. 5A is a schematic diagram of a rear view of a head and chin rest, in accordance with some embodiments of the present invention.

FIG. 5B is a schematic diagram of a front view of the head and chin rest of FIG. 5A, in accordance with some embodiments of the present invention.

FIG. 5C is a schematic diagram of a top view of the head and chin rest of FIG. 5A, in accordance with some embodiments of the present invention.

FIG. 5D is a schematic diagram of a rear perspective view of the head and chin rest of FIG. 5A, in accordance with some embodiments of the present invention.

FIG. 5E is an attachable fabric strap that can be attached to the head and chin rest when extra head movement needs to be prevented, in accordance with some embodiments of the present invention.

FIG. 6A a rear perspective view of an exemplary ophthalmoscopic device, in accordance with some embodiments of the present invention.

FIG. 6B is a front view of another exemplary ophthalmoscopic device, in accordance with some embodiments of the present invention.

FIG. 6C is a rear view of another exemplary ophthalmoscopic device, in accordance with some embodiments of the present invention.

FIG. 7A is a schematic diagram of a top view of a subject's head positioned with an exemplary head and chin rest of FIG. 6A, in accordance with some embodiments of the present invention.

FIG. 7B is a schematic diagram of a top perspective view of a subject's head positioned with an exemplary head and chin rest of FIG. 6A, in accordance with some embodiments of the present invention.

FIG. 7C is a schematic diagram of a side view of a subject's head positioned with an exemplary head and chin rest of FIG. 6A, in accordance with some embodiments of the present invention.

FIG. 7D is a schematic diagram of a front perspective view of a subject's head positioned with an exemplary head and chin rest of FIG. 6A, in accordance with some embodiments of the present invention.

FIG. 8A provides a diagram of a first exemplary optical array, consistent with some embodiments of the present invention.

FIG. 8B provides a diagram of a second exemplary optical array, consistent with some embodiments of the present invention.

FIG. 9 provides a flowchart of an exemplary process for generating and correcting an image, or a series of images, of a retina using mono-directionally or bi-directionally captured retinal image data, consistent with some embodiments of the present invention.

FIG. 10 provides a flowchart of an exemplary process for determining absolute and/or relative movements of the eye and/or retina and providing an indication of same to an operator, consistent with some embodiments of the present invention.

FIG. 11A provides an image of an exemplary retinal reference frame image, consistent with some embodiments of the present invention.

FIG. 11B provides an exemplary non-reference frame image, consistent with some embodiments of the present invention.

FIG. 12A provides a screen shot of a first exemplary graphic user interface (GUI), consistent with some embodiments of the present invention.

FIG. 12B provides a screen shot of a second exemplary graphic user interface (GUI), consistent with some embodiments of the present invention.

FIG. 12C provides a screen shot of a third exemplary graphic user interface (GUI), consistent with some embodiments of the present invention.

FIG. 12D provides a screen shot of a fourth exemplary graphic user interface (GUI), consistent with some embodiments of the present invention.

FIG. 13A is a diagram of a first fixation target image, consistent with some embodiments of the present invention.

FIG. 13B is a diagram of a second fixation target image, consistent with some embodiments of the present invention.

FIG. 13C is a diagram of a third fixation target image, consistent with some embodiments of the present invention.

FIG. 13D is a diagram of a fourth fixation target image, consistent with some embodiments of the present invention.

FIG. 14 provides a flowchart of an exemplary process for generating a composite retinal image and using the composite retinal image to determine a feature or characteristic of a subsequently received image of the retina, consistent with some embodiments of the present invention.

FIG. 15A provides a first image that shows an upper-left field of view of a subject's retina, consistent with some embodiments of the present invention.

FIG. 15B provides a second image that shows an upper-center field of view of the subject's retina, consistent with some embodiments of the present invention.

FIG. 15C provides a third image that shows an upper-right field of view of the subject's retina, consistent with some embodiments of the present invention.

FIG. 15D provides a fourth image that shows a center-left field of view of the subject's retina, consistent with some embodiments of the present invention.

FIG. 15E provides a fifth image that shows a center field of view of the subject's retina, consistent with some embodiments of the present invention.

FIG. 15F provides a sixth image that shows a center-right field of view of the subject's retina, consistent with some embodiments of the present invention.

FIG. 15G provides a seventh image that shows a lower-left field of view of the subject's retina, consistent with some embodiments of the present invention.

FIG. 15H provides an eighth image that shows a lower-center field of view of the subject's retina, consistent with some embodiments of the present invention.

FIG. 15I provides a ninth image that shows a lower-right field of view of the subject's retina, consistent with some embodiments of the present invention.

FIG. 15J provides a photograph of the exemplary composite retinal image of 15K with gridlines superimposed thereon, consistent with some embodiments of the present invention.

FIG. 15K provides a photograph of the exemplary composite retinal image of FIG. 15J without the gridlines superimposed thereon, consistent with some embodiments of the present invention.

FIG. 16 provides a flowchart of an exemplary process for evaluating a strength and/or quality of a detection path signal captured using a scanning laser ophthalmoscope (SLO) and/or generating a set of pre-processed detection path data, consistent with some embodiments of the present invention.

FIG. 17A provides a graph of a radially averaged power spectrum for an image shown in FIG. 17B, consistent with some embodiments of the present invention.

FIG. 17B provides an image of subject's retina, consistent with some embodiments of the present invention.

FIG. 17C provides a first retinal image and a corresponding graph showing results of a mathematical analysis of the first retinal image, consistent with some embodiments of the present invention.

FIG. 17D provides a second retinal image and a corresponding graph showing results of a mathematical analysis of the second retinal image, consistent with some embodiments of the present invention.

FIG. 18 provides a flowchart of an exemplary process for training a machine learning architecture to recognize features of a retinal image, consistent with some embodiments of the present invention.

FIG. 19A is a retinal image with markings of various features superimposed thereon, consistent with some embodiments of the present invention.

FIG. 19B is another retinal image with markings of various features superimposed thereon, consistent with some embodiments of the present invention.

FIG. 20 provides a flowchart of an exemplary process for predicting and/or modeling features of a retinal image, consistent with some embodiments of the present invention.

FIG. 21A provides a first retinal image, consistent with some embodiments of the present invention.

FIG. 21B provides a depiction of a model of the first retinal image of FIG. 21A, consistent with some embodiments of the present invention.

FIG. 21C provides a second retinal image, consistent with some embodiments of the present invention,

FIG. 21D provides a depiction of a model of the second retinal image of FIG. 21C, consistent with some embodiments of the present invention.

FIG. 21E provides a third retinal image, consistent with some embodiments of the present invention.

FIG. 21F provides a depiction of a model of the third retinal image of FIG. 21E, consistent with some embodiments of the present invention.

FIG. 22 provides a flowchart of an exemplary process for using predicted and/or modeled features of a retinal image, or a series of retinal images, to determine characteristics of the retina and/or track retinal/eye motion over time, consistent with some embodiments of the present invention.

FIG. 23 provides a flowchart of an exemplary process for detecting and/or analyzing characteristics of retinal motion over time, consistent with some embodiments of the present invention.

FIG. 24A is photograph of a first retinal image, consistent with some embodiments of the present invention.

FIG. 24B is a depiction of a modified version of the retinal image provided by FIG. 23A, consistent with some embodiments of the present invention.

FIG. 24C provides measurements and graphs of measurements and analysis pertaining to analysis of the modified versions of the retinal images provided by FIG. 23B, consistent with some embodiments of the present invention.

FIG. 24D is photograph of a second retinal image, consistent with some embodiments of the present invention.

FIG. 24E is a depiction of a modified version of the retinal image provided by FIG. 23D, consistent with some embodiments of the present invention.

FIG. 24F provides measurements and graphs of measurements and analysis pertaining to analysis of the modified versions of the retinal image provided by FIG. 23E, consistent with some embodiments of the present invention.

Throughout the drawings, the same reference numerals, and characters, unless otherwise stated, are used to denote like features, elements, components, or portions of the illustrated embodiments. Moreover, while the subject invention will now be described in detail with reference to the drawings, the description is done in connection with the illustrative embodiments. It is intended that changes and modifications can be made to the described embodiments without departing from the true scope and spirit of the subject invention as defined by the appended claims.

### WRITTEN DESCRIPTION

The human eye is constantly in motion even when a subject is fixating on a target (i.e., staring at a fixed target such as an image or a light) because human eyes (an animal eyes with foveal vision) drift and make microcsaccades (i.e., small involuntary jerky movements of the eye) during fixation to maximize visual acuity. Taking a sequence of images of a subject's retina while the subject is fixating on a target over time and/or looking between two targets voluntarily, and/or following a single moving target allows for a determination of a position of one or more retina features (e.g., photoreceptor, blood vessel, fovea, etc.) for each image of the sequence at a particular point in time. Analysis of the position of the retina feature over the sequence of images allows for determinations of how the retina has moved (e.g., direction, speed, velocity, number of microsaccades, etc.) while the subject's eye was fixated on, following, or looking towards a target. The systems and devices disclosed herein provide a robust, compact, and cost-effective system capable of capturing such retinal images and video so that, for example, accurate image-based tracking of the retina during fixational, saccadic, smooth pursuit, and/or microsaccadic eye movements may be performed. In many embodiments, the systems disclosed herein may be configured to for the recording, viewing, measuring, and analyzing temporal characteristics of saccadic fixation and/or smooth pursuit responses when viewing a static and/or moving visual stimulus and identifying metrics and stability of fixation, smooth pursuit, and/or saccades. Subject data is analyzed for microsaccades, metrics quantifying the fixational eye movement (micro-saccades and drift), as well as voluntary saccades in the horizontal and vertical directions and smooth pursuit.

Systems and devices disclosed herein incorporate a series of optical components (e.g., lenses, mirrors, beam splitters, etc.) and an SLO and/or TSLO for obtaining high resolution images, or a series of images (e.g., 1-300 second videos) of a subject's retina, or retinas, when the subject is focusing on a single target (e.g., a light, image, or video) and/or when the subject is voluntarily moving his or her eyes to focus on, for example, two or more fixation targets. At times, the systems disclosed herein utilize a low power laser beam to scan in one and/or two dimensions (e.g., X-dimension and Y-dimension) over the retina. In some embodiments, the scanning of the retina may be bi-directional. The reflected (or returned) light is detected and used to generate a digital image with a computer or electronic imaging device of the subject's retina. In some embodiments, the systems disclosed herein may be monocular and/or binocular systems that incorporate eye tracking and other processes to measure and report fixation and saccadic retinal responses to displayed fixation targets and/or fixation videos. In some embodiments, images obtained by the SLO and/or TSLO may be evaluated for signal quality so that, for example, images that are of low quality, low resolution, and/or noisy may be removed from a set of images that are analyzed according to, for example, one or more processes described herein.

In some embodiments, the present invention may enable, or facilitate, the creation of a relatively large composite retinal image of a subject's retina that may be used as a reference frame for comparison with one or more images of the subject's retina. The composite retinal image may be made using a plurality (e.g., 2, 3, 4, 6, 9, 12, 16, etc.) of smaller images of different retinal regions that are arranged to form a larger, composite, image of the retina. For example, in one embodiment, a series of nine high-resolution retinal images is combined, or integrated, together with a, for example, a 0-0.5 degree overlap to create a single, larger field of view (FOV) reference frame image that may have a FOV of, for example, 8-20 degrees that, in some instances, may be centered on a region of interest (e.g., fovea, blood vessel, or vessel crossing). In some circumstances, this retinal composite image may be used as a reference frame, or image, of the subject's retina. Using a composite retinal image generated as disclosed herein may enable quantification of both horizontal and vertical movement via analysis of retinal structure data and/or measuring a difference of position of retinal structure between the composite retinal image and a later-taken retinal image. At times, the composite image may be used to capture larger and faster motion during retinal-tracking due to its ability to provide more retinal structure to cross correlate with subsequently taken images, or portions thereof.

Additionally, or alternatively, in some embodiments, the systems and devices disclosed herein may include a defocus correction system that may include two or more defocus correction assemblies that may be synchronized with one another. Each of the defocus correction assemblies may include one or a series of two, or more, optical elements (e.g., lenses and/or mirror and/or display) that may be, for example, opto-mechanically controlled to apply a defocus (spherical equivalent) correction of a subject's eye or eyes to the nearest +/−0.25 diopters. A defocus correction that may be applied by each of the defocus correction assemblies may range from, for example, −12 diopters to +12 diopters. A degree of defocus correction applied by one, or all, of the defocus correction assemblies may be entered manually by an operator of the SLO system and/or may be experimentally determined via observing and/or analyzing (by the operator and/or a computer/processor disclosed herein) retinal images until the retinal image quality is optimized (e.g., clearest retinal image and/or the highest signal-to-noise ratio (SNR) retinal image). In some cases, the defocus assemblies may be communicatively, mechanically, and/or electrically coupled to one another so that an adjustment of one defocus assembly may trigger a corresponding correction for another defocus assembly included in the SLO system. At times, this corresponding correction of the two or more of the defocus assemblies may be performed by, for example, automatically scaling and/or focusing light and/or images provided by one or more paths of the SLO system.

In addition, disclosed herein are systems, devices, and methods for training a machine learning architecture and/or a deep neural network to automatically recognize features of a retinal image and/or generate a modeled image that includes features (modeled or actual/measured features) of a retinal image. Initially, this training may involve the use of very high-resolution images of a retina that are manually and/or automatically marked to point out features of interest (e.g., blood vessels, patterns of blood vessels, capillary crossings, blood vessel crossings, damaged areas, photoreceptors, etc.). These marked retinal images may then be input into the machine learning architecture and/or deep neural network to train the machine learning architecture and/or deep neural network to detect and/or recognize features similar to the marked features in other non-marked retinal images and/or generate corresponding renderings of models of the retinal images that show only the features of interest. In some embodiments, these renderings of the retinal-image models may be analyzed to determine characteristics thereof. Often time this analysis includes analyzing a time series of rendered retinal-image models and measuring movement of, and/or changes to, modeled retinal features over time by comparing two or more of the rendered retinal-image models to one another in order to, for example, track eye motion, and/or provide biological information for the diagnosing, prognosing, and/or monitoring of a disease state and/or the vasculature, capillaries, and/or retinal health of the subject's retina.

The head and chin rests disclosed herein are configured to stabilize a subject's head and limit head motion from side to side, vertically, and in a rotational manner during retinal imaging so that, for example, motion artifacts and/or distortion of retinal images caused by the movement of the subject's head may be reduced or eliminated. The head and chin rests disclosed herein may be configured to accept a wide range of head sizes (e.g., children and adults) and shapes, as well as varying inter-pupillary distances. The head and chin rests disclosed herein may be configured so that a subject may place his or her head within the head and chin rest and remove his or her head from the head and chin rest without obstruction and/or without snagging the subject's hair.

Disclosed herein are head and chin rests for an ophthalmoscopic device (e.g., a SLO, TSLO, or OCT system) configured to take high-quality and/or high-resolution images of a subject's retina. The ophthalmoscopic device described herein may utilize a SLO to measure eye motion. On some occasions, the ophthalmoscopic device may be configured for recording, viewing, measuring, and analyzing temporal characteristics of saccadic and fixation responses when viewing a visual stimulus and identifying metrics and stability of fixation. In some embodiments, the ophthalmoscopic devices disclosed herein may be a monocular or binocular device that incorporates eye tracking to measure and report fixation and saccadic responses. The head and chin rests disclosed herein are configured to stabilize a subject's head, and in some cases, may limit the subject's head motion while one or more images of the subject's eye or retina are taken. In some cases, a video (i.e., series of images) of the subject's eye/retina may be taken over time (e.g., 5-600 seconds) and the head and chin rests disclosed herein may be configured to hold the subject's head in a consistent position for the duration of the video. Additionally, or alternatively, the chin and head rests disclosed herein may be configured to hold the subject's head in a consistent position over time so that, for example, a single and/or a series of images of the subject's retina may be taken without the subject's head moving. In some embodiments, the head and chin rests disclosed herein may stabilize a subject's head and/or limit head motion without a locking mechanism.

In some embodiments, the head and chin rest disclosed herein may utilize a strap that wraps around a back of a subject's head and attaches to a left and right side of the head and chin rest. The strap may assist with securely holding the subject's head in place so that, for example, movement (e.g., rotational, linear, etc.) is reduced and/or eliminated.

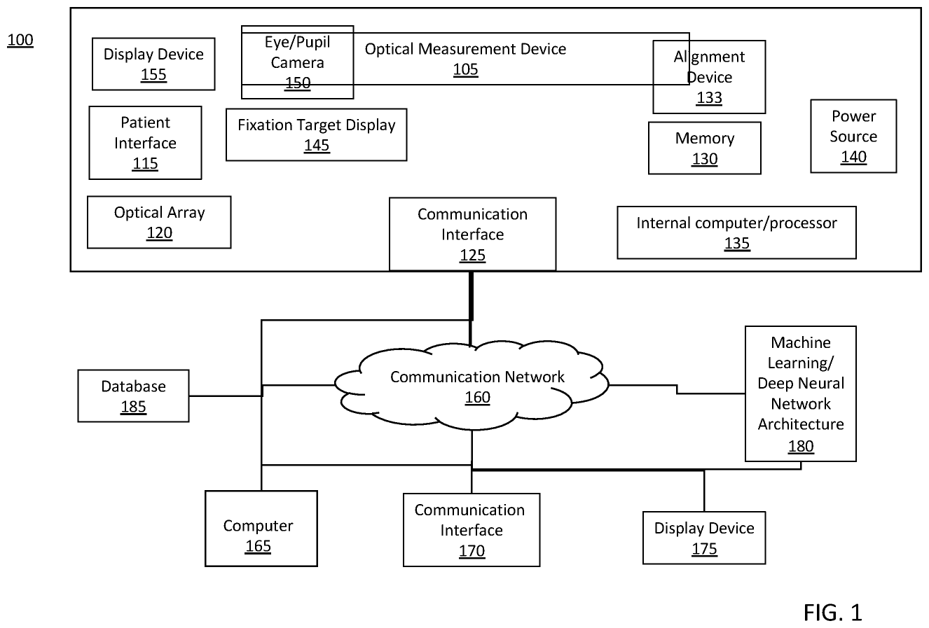

Turning now to the figures, FIG. 1 provides a block diagram of an exemplary SLO system 100 that includes an optical measurement device 105 communicatively coupled to a computer 165 via a communication interface 170 and/or a communication network 160. Communication interface 170 may be any interface configured to enable communication between components of SLO system 100. Exemplary communication interfaces 170 include, but are not limited to, keyboards, speakers, microphones, track pads, communication ports, antennas, transceivers, and other hardware and/or software configured to enable communication between components of SLO system 100. SLO system 100 further includes a display device 175 configured to display information provided by, for example, computer 165 and/or optical measurement device 105. Exemplary display devices include, but are not limited to, display screens and touch sensitive screens configured to electronically display information, such as graphic user interfaces (GUIs) by which a user may interact with computer 165, images, such as images of a retina and/or simplified and/or modified images of a retina as disclosed herein displayed on display device 175, optical measurement device 105 and/or components thereof. In some instances, communication interface 170 and/or display device 175 may be integrated into computer 165 as, for example, a port and/or wireless communication connection but, this need not be the case.

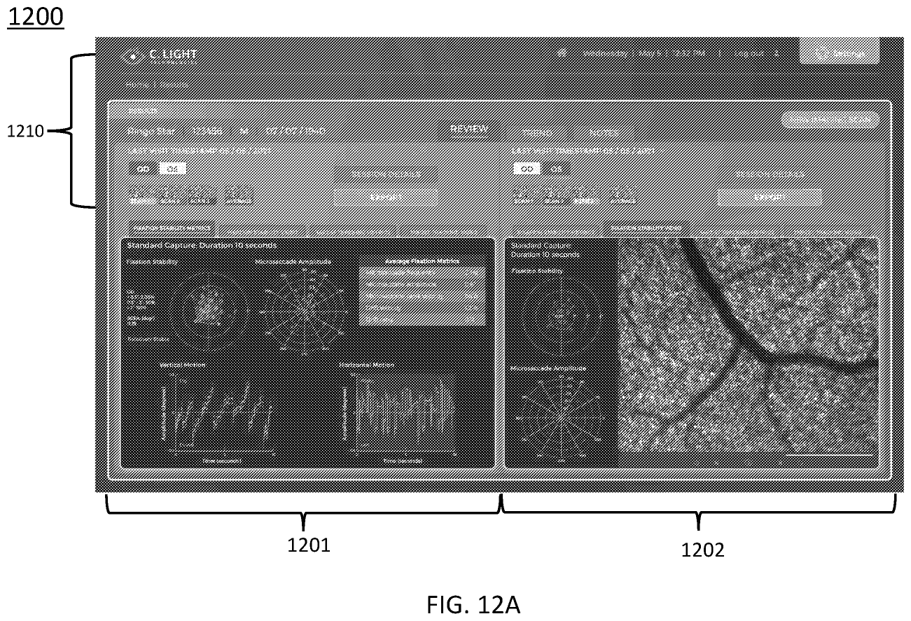

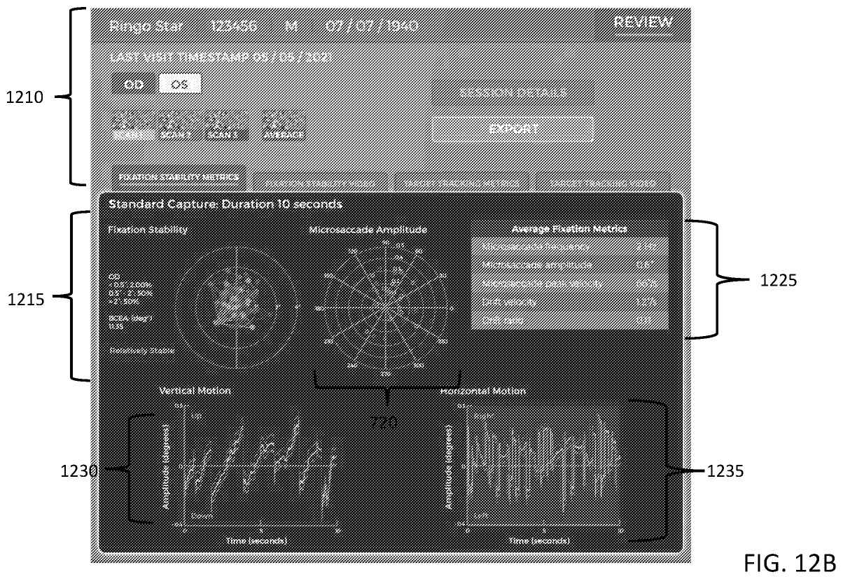

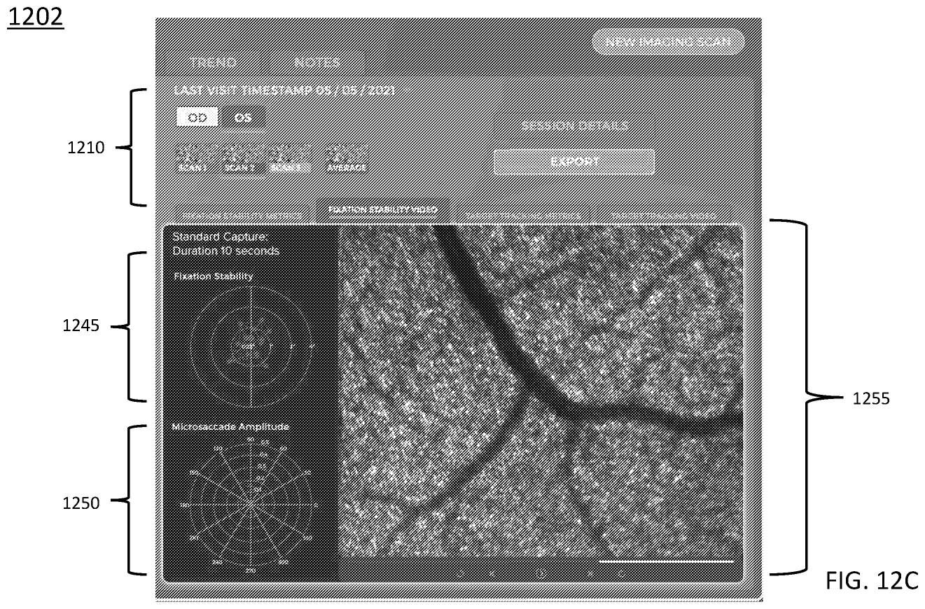

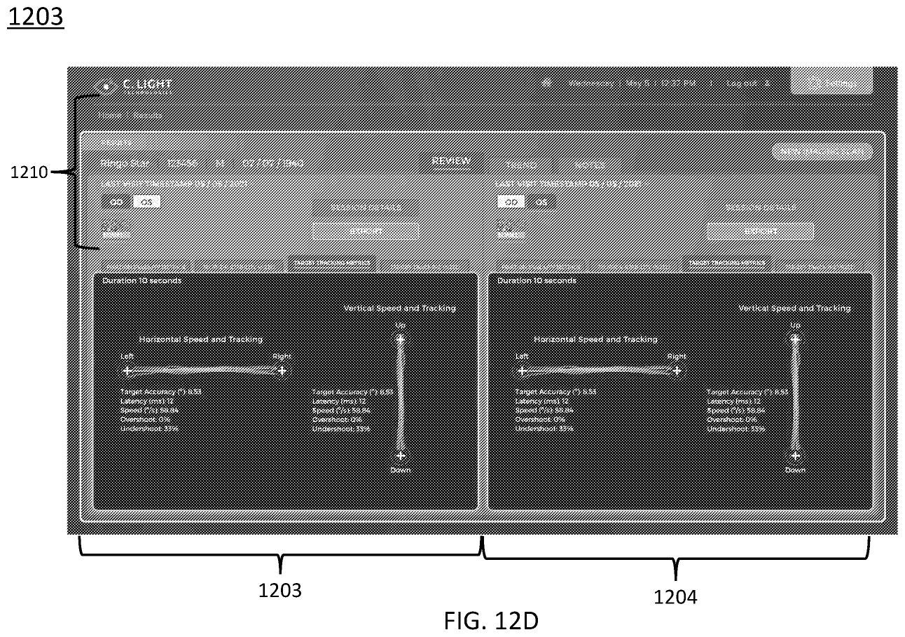

Computer 165 may be any computer system, network of computer systems (e.g., a cloud computing network), and/or device (e.g., application specific integrated circuit (ASIC) and/or field specific gate array (FPGA)) and/or component thereof configured to execute one or more processes, or process steps, described herein. In some cases, computer 165, communication interface 170, and/or display device 175 may be used to operate and/or control optical measurement device 105 and/or information displayed to an operator via, for example, a GUI such as the GUIs provided by FIGS. 12A-12D, discussed herein.

Communication network 160 may be any wired and/or wireless network configured to enable communication between optical measurement device 105 and computer 165. Exemplary communication networks 120 include, but are not limited to, the Internet, LANs, WLANs, mesh networks and Wi-Fi networks. In many instances, communication between optical measurement device 105 and computer 165 may be encrypted or otherwise subject to security protocols that prevent malicious use of information communicated therebetween. In some cases, these security protocols may be compliant with one or more information security regulations (e.g., Health Insurance Portability and Accountability ACT (HIIPA) and General Data Protection Regulation (GDPR).

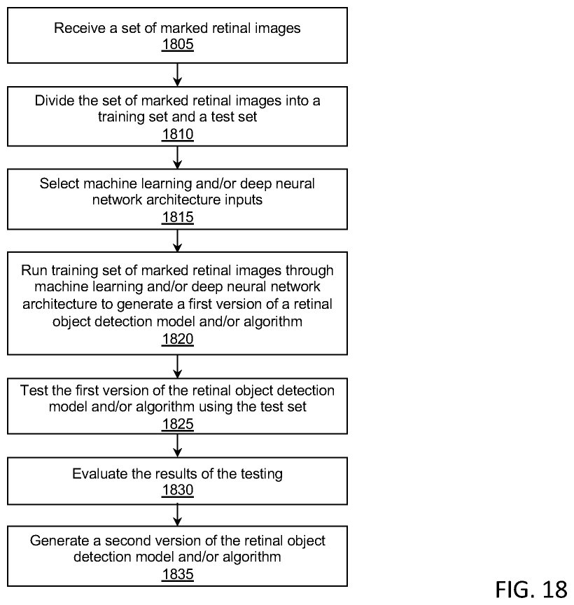

Optionally, system 100 may include a machine learning and/or deep neural network computer architecture 180 that may be configured and programmed to perform one or more of the processes (e.g., process 1800, 2000, 2200, and/or 2300 as shown in FIGS. 18, 20, 22, and 23 and discussed below) described herein. In some instances, machine learning and/or deep neural network computer architecture 180 may be a cloud computing environment that is communicatively coupled to computer 170 via, for example, communication network 160 and/or communication interface 170. In some embodiments, machine learning and/or deep neural network computer architecture 180 may utilize data (e.g., retinal images and/or determinations and/or analysis of retinal images) that are stored in database 185. In some embodiments, machine learning and/or deep neural network computer architecture 180 may be generative adversarial network (GAN) based learning framework.

Optionally, system 100 may include a database 185 communicatively coupled to, for example, optical measurement device 105, computer 165, communication interface, 170, and/or machine learning and/or deep neural network computer architecture 180. Database may be configured and/or programmed to store data obtained by optical measurement device 105 and/or determinations based thereon by, for example, computer 165 and/or machine learning and/or deep neural network computer architecture 180 via, for example, execution of one or more processes described herein. Database 185 may also be programmed and/or configured to store retinal images generated as, for example, described herein and/or one more correlations between a retinal image and characteristics thereof, characteristics of how the retinal image was obtained (e.g., whether the retinal image is part of a set of retinal images taken while capturing fixational eye motion or voluntary saccadic eye motion), and/or characteristics (e.g., age, medical diagnosis, gender, etc.) of a subject whose retina corresponds to a retinal image.

Optical measurement device 105 may include one or more of a patient interface 115, an optical array 120, a communication interface 125, a memory 130, an internal computer/processor 135, a power source 140, a fixation target display 145, an eye/pupil camera 150, and a display device 155. Power source 140 may be any source of electrical power for optical measurement device 105 including, but not limited to, a battery and/or an electrical coupling to an electrical main (a coupling to an electrical cord that may be plugged into an electrical main). Internal computer/processor 135 may be any device, or combination of devices, configured to execute one or more methods disclosed herein. Exemplary components of internal computer/processor 135 include, but are not limited to, electronics cards, ASICs, FPGAs, data acquisition (DAQ) cards, graphical processing units (GPUs), central processing units (CPUs), graphics cards, analog to digital converters (ADC), resonance scanner driver boards, custom signal generation boards, galvanometer driver boards, microelectromechanical (MEMs) driver boards, and/or other devices that may be needed to operate and/or drive optical measurement system 105, system 100, or components thereof. In some embodiments, internal computer/processor 135 may be configured to enable high-bandwidth and/or high-resolution input/output operations that may have a tightly controlled timing and/or frequency of operation. Components of internal computer/processor 135 may be wired and/or wirelessly connected to one another and/or components of system 100 and/or optical measurement system 105.

Memory 130 may be one or more memory devices (e.g., solid state memory devices (SSD), ROM, RAM, and/or combinations thereof) configured to store, for example, instructions for operation of system 100 and/or system components (e.g., optical measurement device 105), instructions for executing one or more processes herein, and/or data gathered by system 100 and/or optical measurement system 105. Communication interface 125 may be any device, or combination of devices, configured to receive information and/or transmit information from, optical measurement device 105. Exemplary communication interface 125 include, but are not limited to, ports, jacks, antenna, near-field communication devices, and the like.

Fixation target display 145 may be configured to display any fixation target configured to focus, direct, and/or guide the subject's fixation while the subject's eye(s) is being tested. In some embodiments, fixation target display may be a small (e.g., a diagonal length of 0.5-5 inches) display device configured to display fixation stimuli that a subject may focus his or her eye(s) on while the subject's eye(s) is/are scanned and/or imaged with an optical array like optical array 120. Exemplary fixation target displays 145 include, but are not limited to, one or more lights, LEDs, display screens, liquid crystal display (LCD) devices, and/or LED display devices. In some embodiments, fixation target display 145 may be a small display screen or device (e.g., liquid crystal display (LCD) or LED display) that displays one or more fixation targets and/or a video including one or more fixation targets.

In some embodiments, fixation target display 145 may operate to display images in black and white and/or color and may have, for example, RGB and/or YCbCr inputs at an appropriate rate (e.g., 0 (when displaying still images) to 150 Hz (when displaying videos)). A fixation target display 145 may be configured to display images at any appropriate resolution (e.g., as 428×240 pixels, 1280×1024 pixels, 1280×720 pixels, 2048×2048 pixels, and/or 2560×1440 pixels).

Exemplary fixation targets 145 include LCDs that, in some cases, may be a high-density transmissive LCDs that have a single crystal silicon backplane, which can vary in both resolution and the diagonal size of the screen in various embodiments. In some cases, a high-density transmissive LCDs may have a resolution of 428×240 pixels, 1280×1024 pixels, or larger. Exemplary sizes for a fixation target display 145 that is embodied as high-density transmissive LCDs is a diagonal length from 0.15-1.45 inches.

Additionally, or alternatively, fixation target display 145 may be an organic light emitting diode (OLED) displays that, in some instances, may include one or more active-matrix organic light emitting diodes (AMOLEDs). At times, an OLED fixation target display 145 may operate with a single crystal silicon transistor concept. An exemplary resolution and/or size for an OLED fixation target display 145 ranges from a resolution of 1280×720 pixels with a 0.4-1 inch diagonal length to a resolution of 2048×2048 pixels with a 0.0.5-2 inch diagonal length.

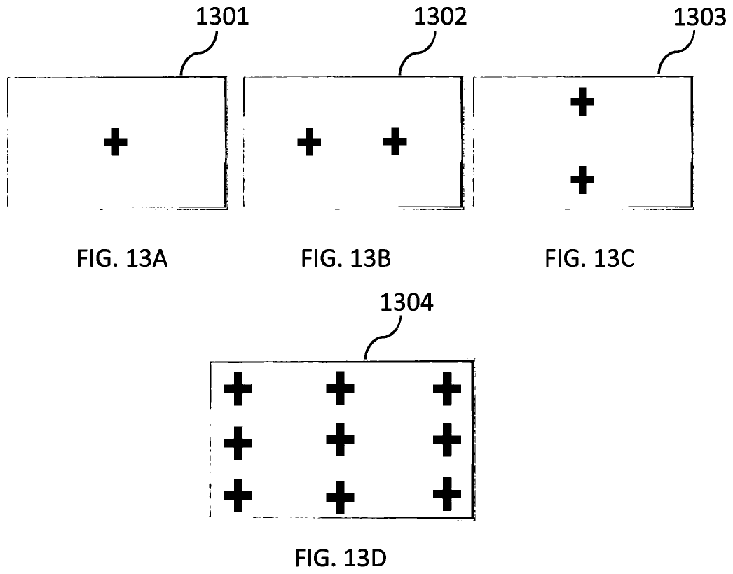

Additionally, or alternatively, a fixation target display 145 may be a ferroelectric liquid crystal on silicon (FLCoS) display. FCLOS displays offer spatial light modulation (SLM), amplitude modulation (AM), and/or binary phase modulation (BPM) that may be programmable with a 2-dimensional diffraction grating. Additionally, or alternatively, use of a FLCoS display for fixation target display 145 also provides for time domain imaging (TDI), which can help stimulate eye motion with videos designed and/or provided by the software. In some cases, use of a FLCoS display as for fixation target display 145 also provides the ability to computer-generated holograms using, for example, the binary phase modulation method. Use of computer-generated holograms may be helpful when interaction between the eyes and the brain. A FLCoS fixation target display 145 may have a resolution of, for example, 2048×2048-pixels and/or 2560×1440-pixels with a 0.5-2.5 inch diagonal length. A FLCoS fixation target display 145 may have both RGB and YCbCr inputs. Exemplary images that may be provided by fixation target display 145 are provided by FIGS. 13A, 13B, 13C, and 13D.

A position of a displayed fixation target may be stationary and/or move over time. In some embodiments, fixation target display 145 may receive instructions regarding what and/or when to display a fixation target from, for example, internal computer/processor 135 and/or computer 165. Exemplary fixation targets include, but are not limited to, an image (e.g., a crosshair, a set of crosshairs, a graphic (e.g., circle, line, or set of circles and/or lines), and a photograph), and/or a series of images (e.g., a movie of a still and/or moving graphic, object, and/or set thereof)). At times, fixation target display 145 may be configured to display images and/or videos that include augmented reality, image fusion, simulation, and/or vision and/or brain training processes. In some embodiments, a fixation target may be a set of images and/or videos configured to assess, for example, fixational eye motion, smooth pursuit, saccades, and/or microsaccades responsively to displayed fixation targets.

In some embodiments, fixation target display 145 may be configured to display fixation stimuli responsively to an instruction from, for example, a processor like internal computer/processor 135 and/or computer 165. Additionally, or alternatively, the fixation target display 145 may be configured to cooperate with a computer driver board (not shown) that may provide one or more instructions regarding fixation stimuli to be displayed by fixation target display 145. The computer driver board may be configured to connect to and/or cooperate with operational software through, for example, internal computer/processor 135 and/or computer 145 in order to receive instructions and/or other parameters for operation such as control for color space conversion, contrast, brightness, and gamma correction of the fixation stimuli provided to fixation target display 145. Exemplary stimuli that may be displayed on fixation target (responsively to instructions from the processor and/or computer) include, but are not limited to, (1) a static image or target, (2) a set of static images/targets or (3) a series of images, or targets, displayed as a video. The series of images, or targets, may be displayed by fixation target display 145 at a rate of, for example, 20-180 Hz.

In some embodiments, an image, or series of images, displayed by fixation target display 145 may be configured for a field of vision for a subject's eye. Additionally, or alternatively, an image, or series of images, displayed by fixation target display 145 may be configured to elicit a response (e.g., eye movement) from the subject in order to track the response and, in some cases, compare to one or more baseline responses to, for example, diagnose a disease and/or track disease progression.

Eye/pupil camera 150 may be an optical instrument (e.g., lens, set of lenses, mirror, set of mirrors, and/or window) configured to allow an operator of optical measurement device 105 to see and optimally align the subject's eye and/or pupil with optical array 120 and/or components thereof by way of, for example, a display of the subject's eye/pupil on display device 155. In some embodiments, fixation target display 145 may be configured to have a center point configured for alignment with the subject's fovea during, for example, an eye and/or pupil alignment procedure performed by an operator using, for example, eye/pupil camera 150. Fixation target display 145 may be configured to have a plurality (e.g., 3, 6, 9, 12, etc.) of positions, or locations, and a center point aligned with the subject's fovea. The remaining positions on the fixation target display 145 may be configured to allow for a desired (e.g.,) 5-40° field of view (FOV) of the retina. In some cases, the images may be configured with a desired (e.g., 0.1-1°) overlap between images.

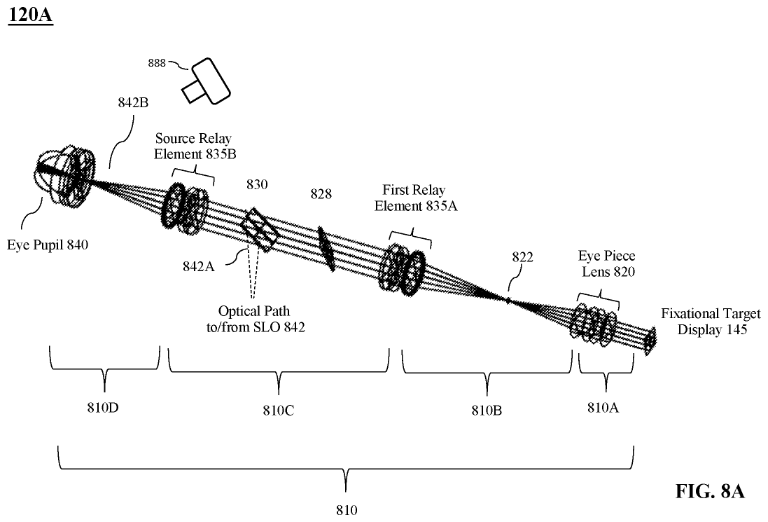

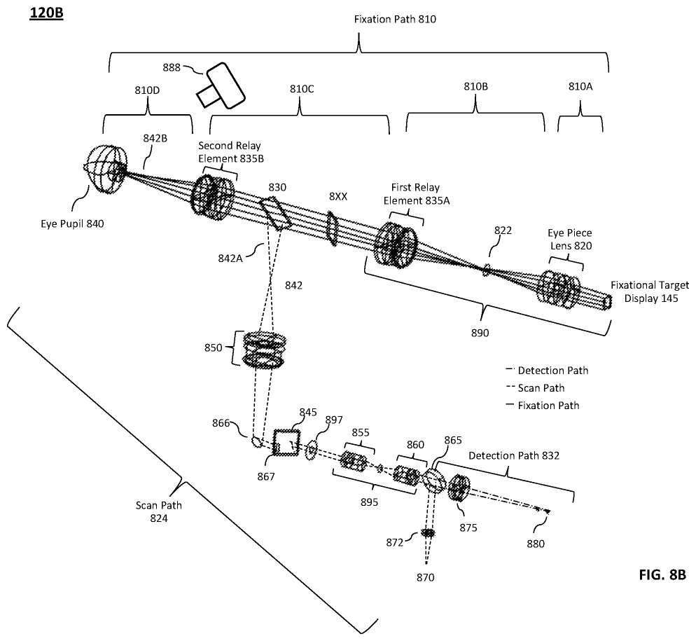

Optical array 120 includes a well-aligned system of relay elements, lenses, scanners, beam-splitters, acousto-optic modulators (AOM) configured to selectively attenuate a beam of scanning radiation, and/or opto-mechanical components that deliver light from a source (e.g., a super luminescent diode fiber) to the subject's eye. The light goes through a series of achromatic doublet lenses that relay the input beam onto a series of scanners (e.g., a resonance scanner, a galvanometer scanner, a MEM mirror and/or a scanning device, etc.) and onto the subject's eye. Light is then reflected from the human retina to a beam-splitter that directs the light to the detector for data collection and analysis. Optical array may be configured to have one or more automatic operations including, but not limited to auto-alignment, auto-focus, auto-exposure, and/or auto-capture and may have a focus adjustment range between −12 D to +12 D. Further details of exemplary optical arrays consistent with the present invention are provided in FIGS. 8A and 8B and discussed below.

Optionally, optical measurement device 105 may include an alignment device 133 configured and arranged to assist with aligning a subject's eye/pupil with optical array 120. Exemplary alignment devices 133 include, but are not limited to, cameras, apertures, and lenses.

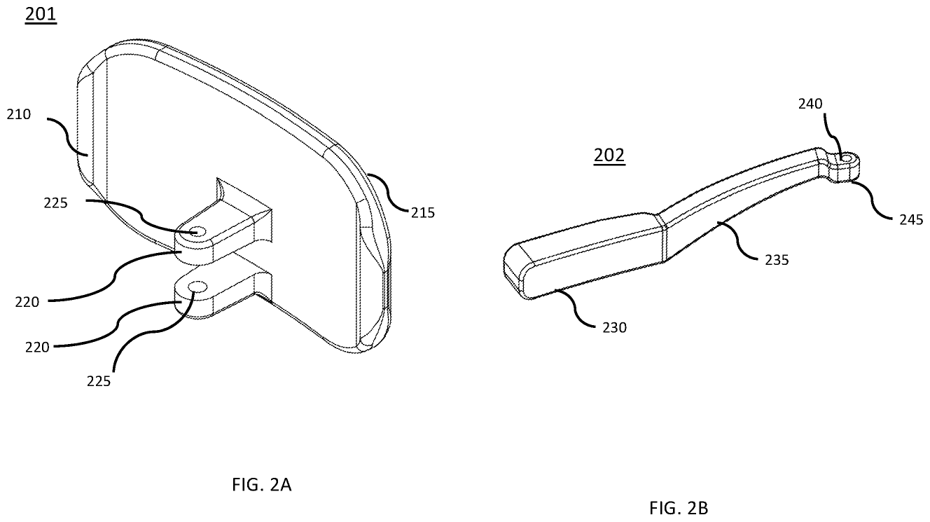

FIG. 2A is a schematic diagram of a rear view of a temple pad 100 for an ophthalmoscopic device (e.g., a SLO, TSLO, or OCT system) configured to take high-quality images of a subject's retina. Temple pad 100 has a front surface 115 and a back surface 110 from which two extensions 120 project as shown. Each extension 120 includes a hole 125 bored vertically (as oriented in FIG. 1) through extensions 120. Temple pad 100 may be curved to, for example, approximate a curvature of a subject's head near the temple area. A length of extension 120 may be configured to position temple pad 100 at a distance (e.g., 0.7-2.5 cm) away from a head portion of a head and chin rest that prevents the trapping of hair between temple pad 100 and the head portion of a head and chin rest.

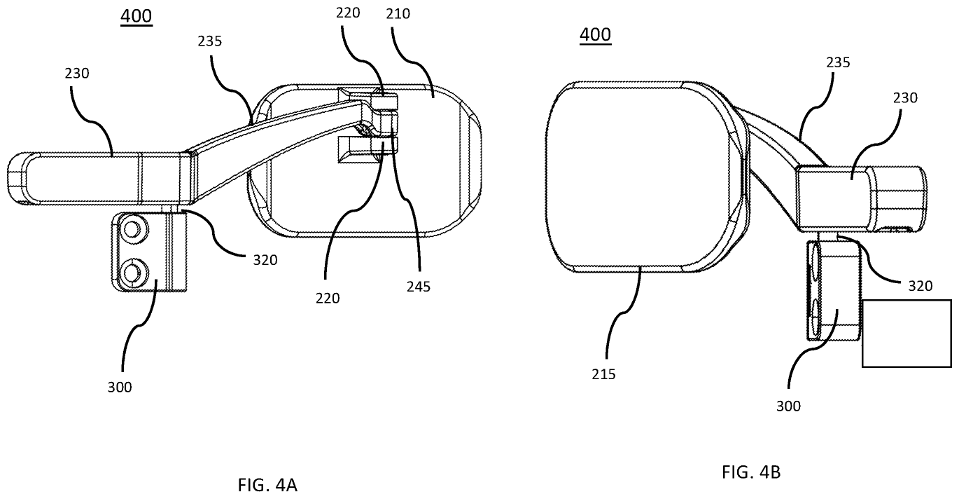

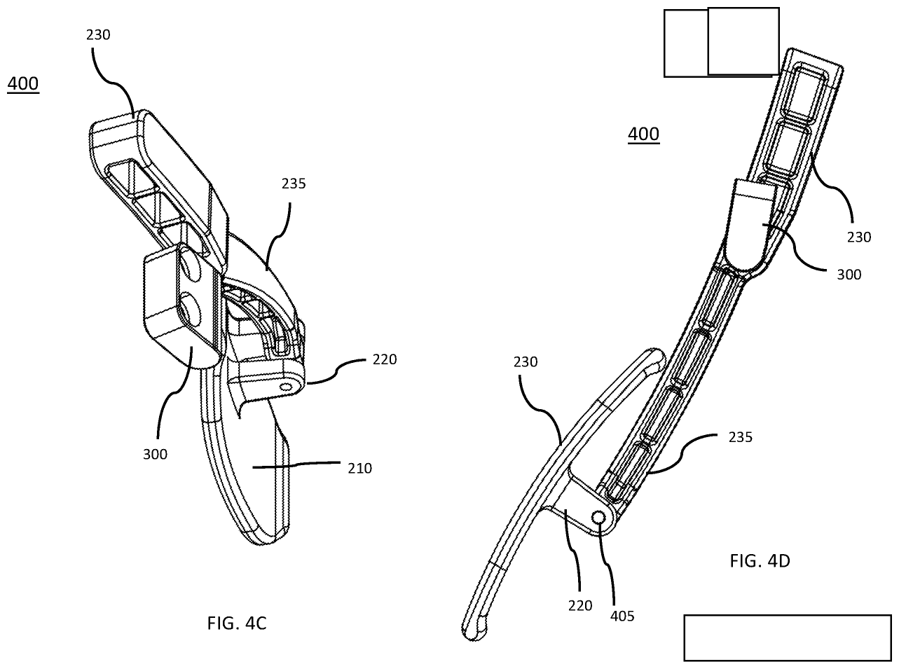

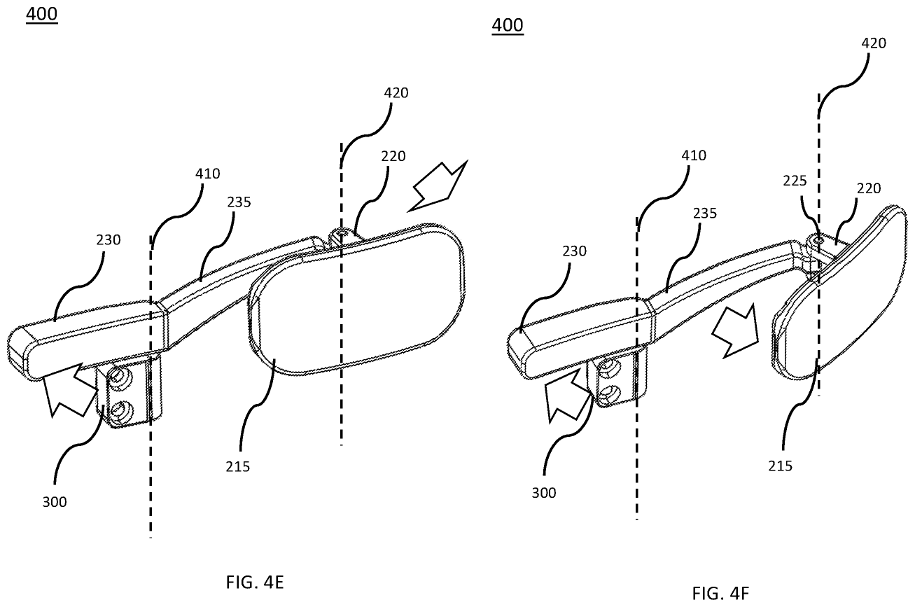

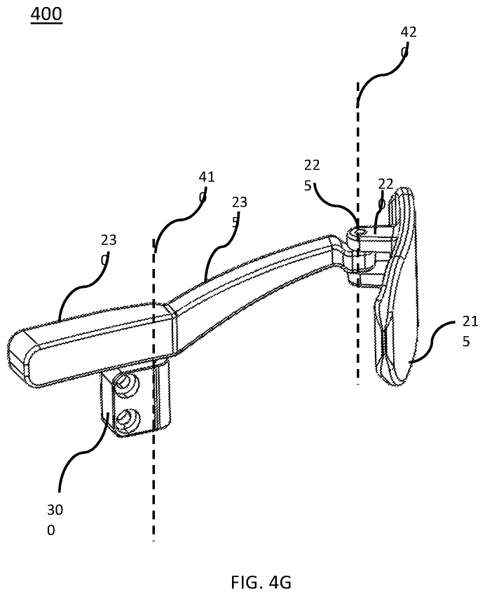

FIG. 2B is a schematic diagram of a front perspective view of a temple arm 200 that may be configured to couple to and cooperate with temple pad 100 as part of a temple pad assembly (as shown in FIGS. 4A-4D and discussed below). Temple arm 200 includes a tab 205, an arm 210, a temple coupling extension 215, and a temple arm hole 220. Temple arm 200 may be configured so that temple coupling extension 215 may be inserted between extensions 120 and temple arm hole 220 aligns with temple pad holes 125 when temple coupling extension 215 is inserted therein. Following insertion of temple coupling extension 215 between extensions 120 and alignment of temple pad holes 125 with temple arm hole 220 is achieved, a coupling mechanism (e.g., a pin or screw) may be inserted into aligned temple pad holes 125 and temple arm hole 220 to fasten them together into a temple pad assembly that may be configured to rotate freely about an axis caused by the coupling mechanism.

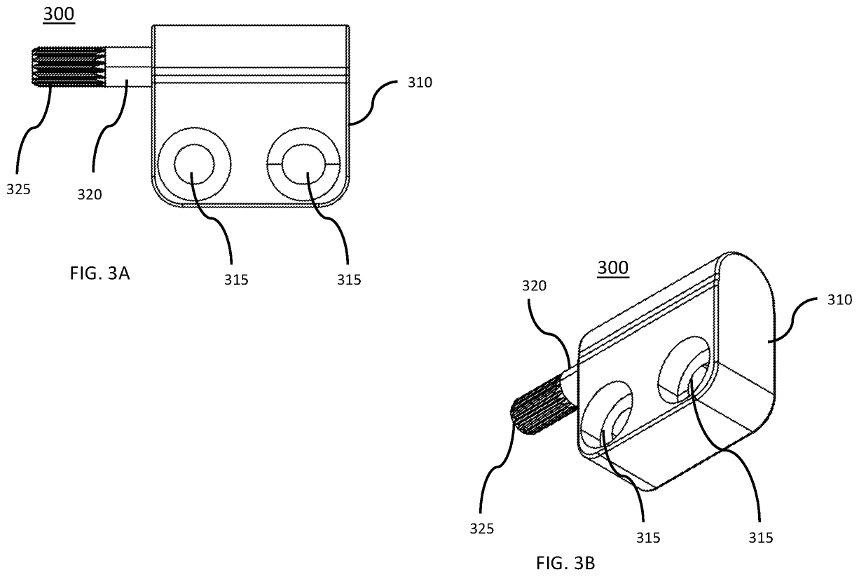

The temple pad assembly may be attached to a head and chin rest frame via, for example, a torque hinge. FIG. 3A is a schematic diagram of a front plan view of an exemplary torque hinge 300 and FIG. 3B is a schematic diagram of a front perspective view of torque hinge 300. Torque hinge 300 includes a body 310 with two holes, or apertures, 315 configured to accept an attachment mechanism (e.g., a screw or pin) by which torque hinge 300 may be attached to a head and chin rest frame. Body 310 also includes a cylindrical extension 320 that includes an engagement mechanism 325 embodied, in this instance, as a plurality of grooves, or extensions, that encircle, or surround, a portion of cylindrical extension 320. In the embodiment of FIG. 3A, engagement mechanism 325 includes a plurality of groves that are parallel in orientation to extension 320. In some embodiments, engagement mechanism 325 may be embodied as a keyed shape (e.g., a “D”-like shape) configured to fit within a corresponding hole or opening in tab 205. This can also be embodied as a keyed shape such as a D without the grooved edges. Torque hinge 300 may be, for example, a single direction torque hinge or a bi-directional torque hinge.

FIGS. 4A-4D provide various views of an exemplary temple pad assembly 400 that includes temple pad 100, lever arm 200, and torque hinge 300 assembled together as shown. In particular, FIG. 4A is a schematic diagram of a rear view of a temple pad assembly, FIG. 4B is a side view of temple pad assembly 100, FIG. 4C is a rear perspective view of a temple pad assembly 400, and FIG. 4D is a bottom view of a temple pad assembly 400. As may be seen in FIG. 4A, temple coupling extension 215 is positioned between two extensions 120 so that extension holes 125 align with temple arm holes 220 and a pin, or another attachment/securing mechanism (not shown) may be inserted into a first extension hole 125, through temple arm hole 220, and through a second extension hole 125, thereby creating a temple pad hinge. Temple pad 100 may be configured to articulate around the pin, or attachment mechanism, holding temple coupling extension 215 and two extensions 120 together. This articulation may be configured to accommodate or match, a shape of a subject's head positioned proximate to temple pad assembly 400 when the head and chin rests disclosed herein are in use.

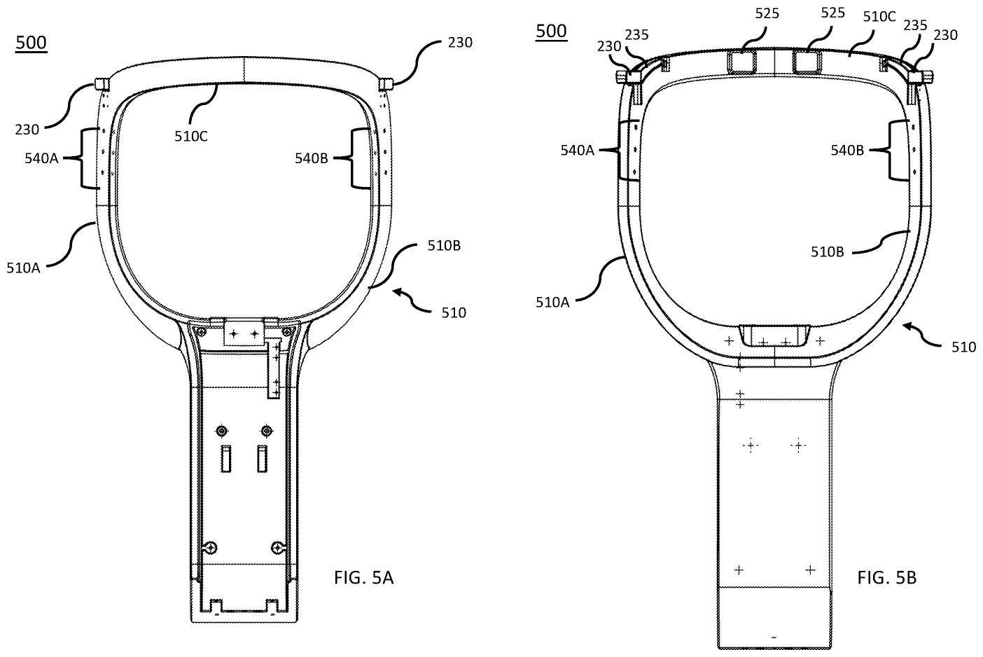

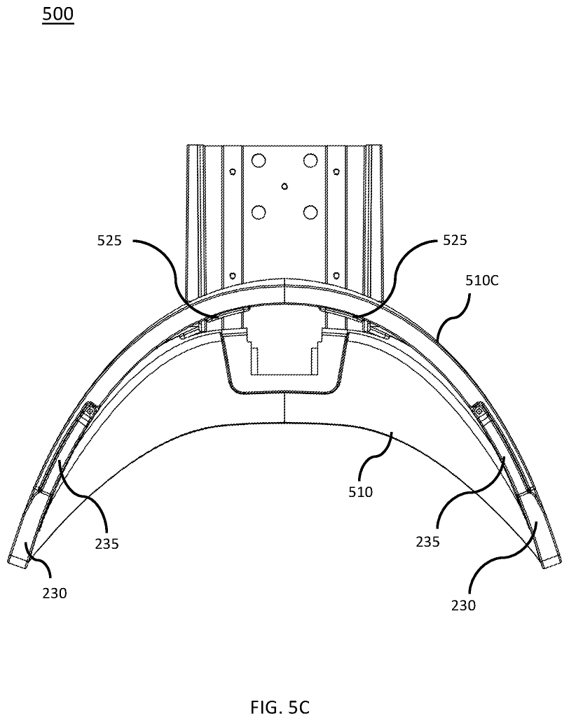

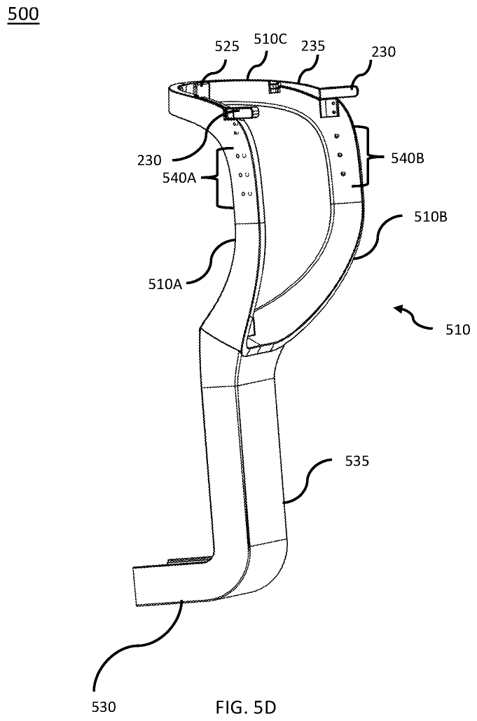

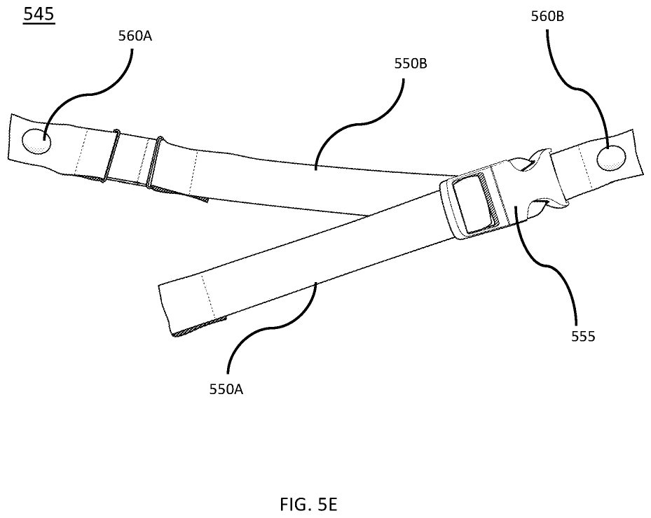

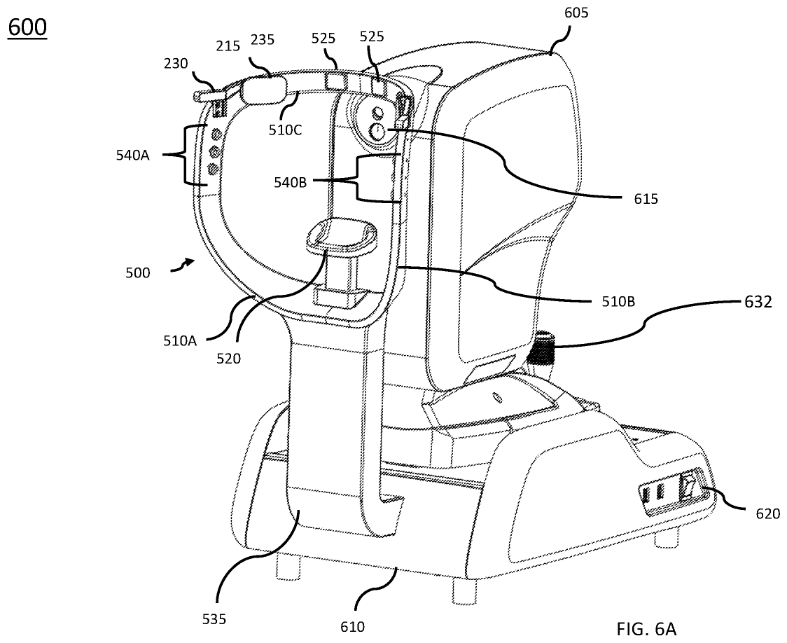

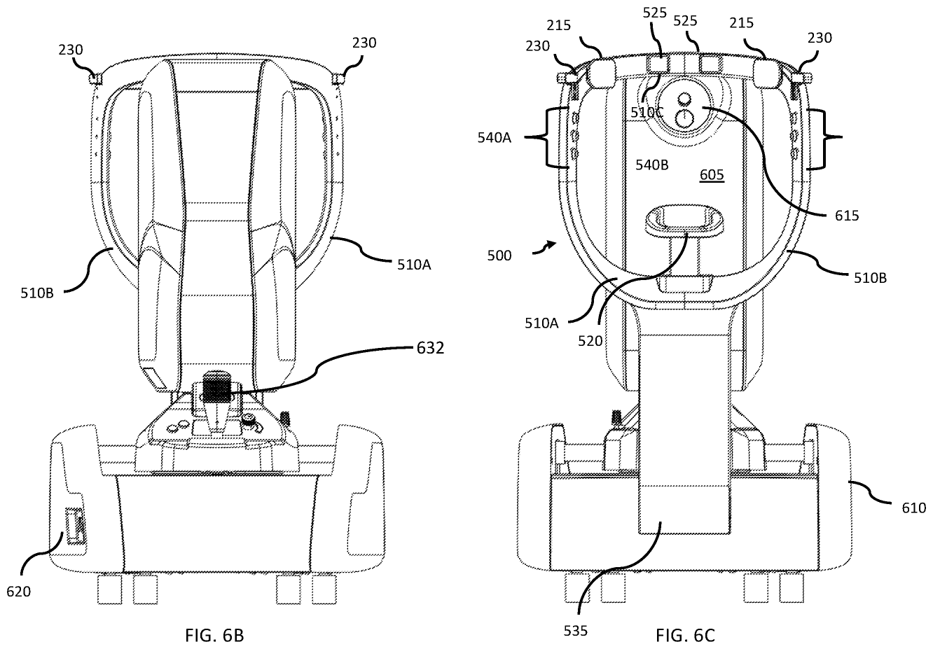

In addition, grooved edge 325 of torque hinge 300 may be inserted into an opening, or hole, positioned on an underside (as oriented in FIG. 4A) of tab 205 so that only extension 320 may be visible. Torque hinge 300 may be configured to attach to a head and chin rest frame such as head and chin rest frame 510 discussed below with regard to FIGS. 5A-6B via, for example, with two or more screws or mounting hardware (not shown). When arranged in this configuration, torque hinge 300 may be configured to resist force exerted thereon (e.g., 2-5 in-lb.) and may, therefore, assist with holding temple pad assembly 400 against the subject's head, which may reduce motion of the subject's head. An approximate distance between torque hinge 300 and temple pad hinge is 0.8-80 mm (FIGS. 4A-4G).

FIGS. 4E-4G are schematic diagrams of temple pad assembly 400 transitioning from a retracted position, as shown in FIG. 4E to an open position as shown in FIG. 4G. While in a retracted position as shown in FIG. 4E, back surface 110 of temple pad 100 may be positioned relatively close to arm 210 so that an orientation of temple pad 100 is approximately parallel to an orientation of arm 210. FIG. 4E also shows a torque-hinge axis of rotation 410 positioned at a hinge created where grooved edge 325 is inserted into tab 205 and a temple pad axis of rotation 420 positioned along a temple pad hinge. To transition from the retracted position of FIG. 4E to a partially open position as shown in FIG. 4F, a force is applied to tab 205 in the direction shown in FIG. 4E and this force is translated via torque hinge/torque-hinge axis of rotation 410 to arm 210 so that temple pad 100 rotates around temple pad hinge/temple pad axis of rotation 420 as shown in FIG. 4E to arrive at the orientation of the partially open position as shown in FIG. 4F. For temple pad assembly to arrive at a fully open, or extended state, force would continue to be exerted on pad 205 so that arm 210 continues to rotate around torque-hinge axis of rotation 410 and that motion is translated to temple pad 100 as it rotates around temple pad hinge/temple pad axis of rotation 420 as shown in FIG. 4G. When installed into a head and chin rest frame, temple pad assembly 400 may have temple pad retracted into the head and chin rest frame while in the retracted position of FIG. 4E and extended away from the head and chin rest frame (and toward a subject's head) while in the extended, or open, position of FIG. 4G.

FIG. 5A is a schematic diagram of a rear view of a head and chin rest 500 that includes a head and chin rest frame 510. Head and chin rest frame 510 includes a left vertically oriented arm 510A, a right vertically oriented arm 510B, and a top horizontally oriented arm 510C. Left vertically oriented arm 510A and right vertically oriented arm 510B include a first and a second array of attachment mechanisms 540A and 540B, respectively, configured for cooperation with a corresponding attachment mechanism provided by a head-restraining device (e.g., a strap or net) such as an exemplary head-restraining device 545 shown in FIG. 5E and discussed below. First and second array of attachment mechanisms 540A and 540B may include, for example, 1-6 attachment mechanisms configured to accommodate differently sized heads and/or different types of head-restraining devices that may have, for example, 2, 4, or 6 corresponding attachment mechanisms. Exemplary attachment mechanisms included in first and/or second array of attachment mechanisms 540A and 540B include, but are not limited to, a snap, a button, a hook, a loop, interlocking fasteners, a tab, an adhesive, and/or VELCRO™.

Head and chin rest 500 includes a chin rest 520 that may be configured to articulate up and down (as oriented in the figure) so that a subject's head may be correctly positioned within head and chin rest 500 and/or the subject's eye(s) may be aligned with retinal imaging hardware (e.g., a camera or target image display device). Head and chin rest 500 also includes temple pad assembly 400 of which tab 205 may be seen in FIG. 5A. Chin rest frame 510 may be configured to be attached to an ophthalmoscopic device (e.g., a SLO, TSLO, or OCT system) as discussed herein with reference to, for example, FIG. 6.

Chin rest 520 may be, for example, a platform configured to comfortably hold a subject's chin in place and may be mechanically coupled to a motor configured to move chin rest up and down so that subjects of different sizes may be correctly positioned in head and chin rest 500. In some embodiments, chin rest 520 may be configured to support 2-15 pounds dynamically (as chin rest 520 transitions up and down) and up to 20 pounds statically while the subject is at rest while having his or her eye(s) imaged.

FIG. 5B is a schematic diagram of a front view of head and chin rest 500 and shows a portion of the head and chin rest into which a subject may place his or her face so that the subject's chin rests upon chin rest 520 and their forehead abuts and/or rests upon two forehead pads 525 positioned on an interior surface of top horizontally oriented arm 510C. Temple arm assembly 200 may also be seen in FIG. 5B, which shows arm 210 and tab 205 attached to the left and right (as oriented in the figure) sides of horizontally oriented arm 510C.

Head and chin rest frame 510 may be curved as may be seen in the top view of FIG. 5C and the rear perspective view of FIG. 5D. A shape and size of the curvature of head and chin rest frame 510 may be configured so that head and chin rest frame 510 wraps partially around a subject's head inserted therein.

When in a retracted position as shown in FIG. 5C, temple arm 200 may retract into head and chin rest frame 510. While in the retracted position, relative positions between temple arm 200 and head and chin rest frame 500 may be sufficient to prevent hair from entering the space between temple arm 200 and head and chin rest frame 500 and getting caught therein.

Temple arm 200 may be configured to articulate from a retracted position to an extended position around a fulcrum at the temple pad hinge. This articulation may be caused by a technician's and/or user's application of force to tab 205 and may cause temple pad 100 to extend away from head and chin rest frame 510 toward the subject's head until temple pad 100 is in contact with the subject's head. While in contact with the subject's head, temple pad 100 may exert force upon and/or passively resist movement of the subject's head thereby stabilizing the subject's head while his or her eye/retina is being imaged.

FIG. 5E is a photograph of an exemplary head-restraining device 545 that may be configured to wrap around a back of subject's head and attach to one or more of first and second arrays of fastening mechanisms 540A and 540B of left vertically oriented arm 510A and right vertically oriented arm 510B, respectively, thereby securely holding the subject's head in place while his or her eye/retina are being imaged. The exemplary head-restraining device 545 shown in FIG. 5E is embodied as a strap with a first side 550A and a second side 550B that are joined by a holder 555 that has an adjustable position relative to first side 550A and second side 550B so that an overall size/length of exemplary head-restraining device 545 may be adjusted. Exemplary head-restraining device 545 further includes a first attachment mechanism 560A and a second attachment mechanism 560B configured to cooperate with one or more corresponding attachment mechanisms of first and second array of attachment mechanisms 540A and 540B to attach exemplary head-restraining device 545 to head and chin rest frame 510 thereby securing a subject's head within head and chin rest frame 510 so that their eye/retina may be imaged without the subject's head movement harming image quality.

FIG. 6A is a front perspective view of an exemplary ophthalmoscopic device 600, such as a SLO, TSLO, or OCT system, that incorporates head and chin rest 500 and/or components thereof such as a set of temple pad assemblies 400 that include temple pads 115, temple arms 210, tabs 205, left vertically-oriented arm 510A, a right vertically-oriented arm 510B, a top horizontally-oriented arm 510C, chin rest 520, temple pads 525, optical array housing 605 and an ophthalmoscopic device base 610. Optical array housing 605 may include one or more components (e.g., lenses, cameras, etc.) configured to capture an image of the subject's eye/retina.

Head and chin rest 500 may be configured and/or arranged to position the subject's head, and more particularly the subject's eye, proximate to an optical head opening 615 in optical array housing 605 so that the subject's retina may be viewed and/or imaged by optical components housed in optical array housing 605. Optical array housing 605 contains optical components (e.g., an optical array) for delivering, collecting, and measuring the light in the system and/or reflected from the subject's eye/retina. The optical components contained in optical array housing 605 may direct light into the subject's eye and detect light reflected from there through optical head opening 615. Optical array housing 605 may also contain electronics and other devices used to direct light into and/or gather and/or process light reflected from the subject's eye/retina. These additional components include, but are not limited to, a resonance scanner, a resonance scanner driver board, a galvanometer signal generation board, a galvanometer driver board, a galvanometer, an acousto optic modulator (AOM), an avalanche photo diode (APD) or photomultiplier tube (PMT) detector, a miniature monitor/fixation target, a video screen driver board, and/or a pupil camera.

Ophthalmoscopic device base 610 may provide a mechanical stability base 610 that facilitates the stability of ophthalmoscopic device 600 and cooperates with head and chin rest 500 and/or chin rest 520, to reduce, or minimize, movement of ophthalmoscopic device and/or the subject's head and/or subsequently the eye/retina. Ophthalmoscopic device base 610 may also house, for example, a power source, a light source (super luminescent light emitting diode (SLD)), a computer memory device (solid-state drive (SSD)), an internal computer/processor, and analog to digital converter (ADC), and/or a communication interface in the form of an array of input/output ports 620. Ophthalmoscopic device 600 may include a controller 632 configured to control a position of as, for example, a joystick.

FIG. 6B is a front perspective view of an exemplary ophthalmoscopic device 601, such as a SLO, TSLO, or OCT system, that incorporates head and chin rest 500 and/or components thereof. Ophthalmoscopic device 601 is similar to ophthalmoscopic device 600, and it also includes a positioning device 630 that is configured to move optical array housing 605, head and chin rest 500, and/or chin rest 520 (and therefore a subject's head/eye) in the X-, Y-, and/or Z-dimensions via a user's interaction with positioning device 630. In some embodiments, positioning device 630 may move optical array housing 605 in the X-, Y-, and/or Z-dimensions and/or chin rest 520 in the Z-dimension.

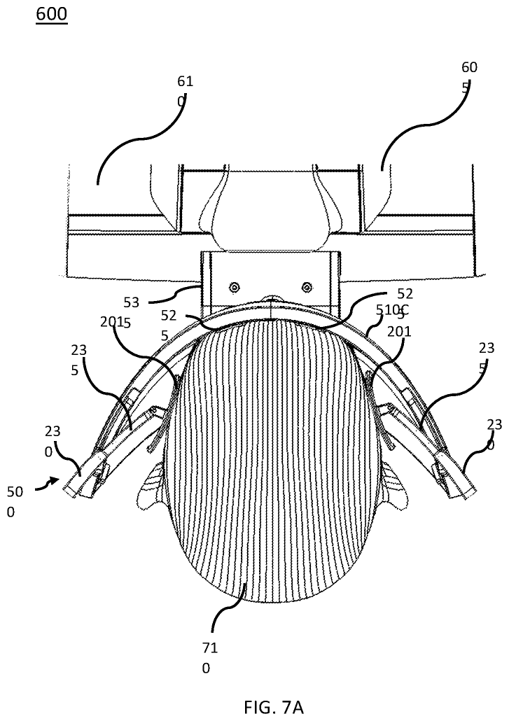

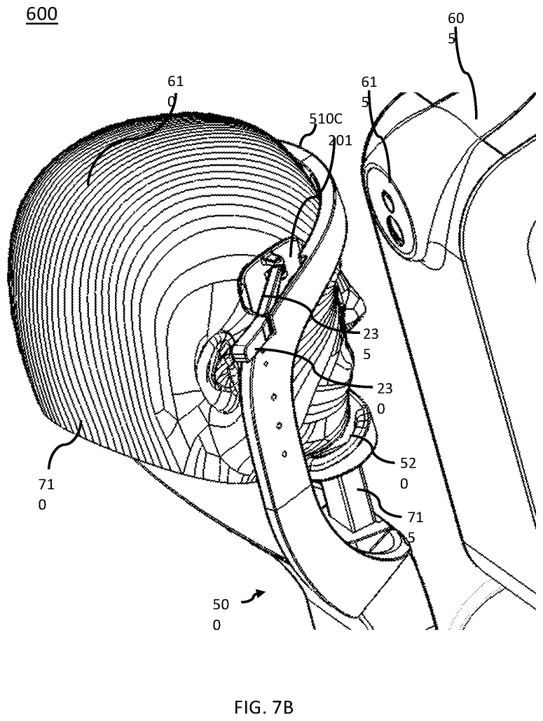

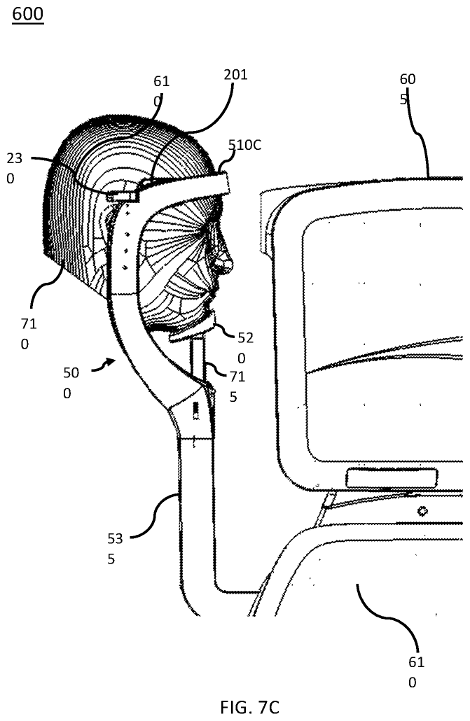

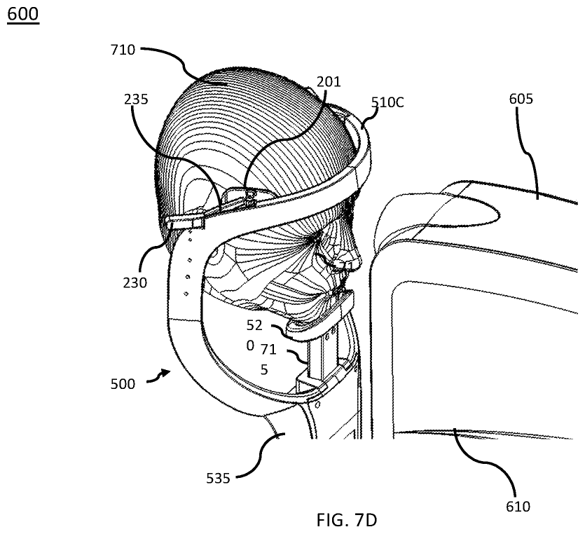

FIGS. 7A-7D are schematic diagrams of an abstraction of subject's head 710 positioned with an exemplary head and chin rest 600 coupled to, for example, exemplary ophthalmoscopic device 600 and/or 601. In the top view of FIG. 7A, an abstraction of subject's head 710 is shown positioned within head and chin rest frame 500 with a temple pad 100 in an open, or an extended, position/configuration as shown in, for example, FIG. 4G so that temple pads 100 are positioned proximate to and/or are abutting subject's head 710 as shown. When in this position, movement of temple pads 100 into a retracted state (i.e., toward head and chin rest frame 500) may be resisted by torque hinge 300 thereby holding subject's head 710 in position and limiting movement thereof. FIG. 7B is a rear-side perspective view of subject's head 710 positioned within head and chin rest frame 500 and it also shows how subject's head 710 is aligned with optical head opening 615 in optical array housing 605 so that the subject's eye and/or retina may be scanned, or imaged, using, for example, an optical array (not shown) contained in optical array housing 605. FIG. 7B also shows how chin rest 520 has been extended away/up from head and chin rest frame 500 via an extension 715 that is configured to raise and lower chin rest 520 so that subject's head 710 may be properly positioned/aligned with optical head opening 615.

FIG. 7C is a side view of subject's head 710 positioned within head and chin rest frame 500 and it also shows how subject's head 710 is aligned with optical array housing 605 that is coupled to ophthalmoscopic device base 610 so that the subject's eye and/or retina may be scanned, or imaged, using, for example, an optical array (not shown) contained in optical array housing 605. FIG. 7C also shows how chin rest 520 is extended away/up from head and chin rest frame 500 via extension 715. FIG. 7D is a front-side perspective view of subject's head 710 positioned within head and chin rest frame 500 and it also shows how subject's head 710 is aligned with optical array housing 605 so that the subject's eye and/or retina may be scanned, or imaged, using, for example, an optical array (not shown) contained in optical array housing 605.

In some embodiments, the head and chin rests disclosed herein may include a locking mechanism configured to lock one or more components (e.g., temple pad assembly 400) thereof in place. The locking mechanism may be active or passive and may be configured to resist movement of the subject's head. Additionally, or alternatively, temple pads 100 may be rigidly connected to arm 210 so that temple pad assembly 400 only rotates around torque hinge 300/torque-hinge axis of rotation 410.

Although the embodiments disclosed herein are mechanical in nature, that need not be the case and one or more functions of the head and chin rests disclosed herein may be electronically activated (via, for example, pushing a button or selecting an icon provided by a graphical user interface of a software application) and/or performed via motors (that in some cases may be coupled to a drive cable), or other electronic devices. At times, one or more components of the systems and devices disclosed herein may be actuated via a drive cable, a pneumatic device, or other mechanisms that provide actuation force. This actuation force may be applied to the one or more components of the systems and devices disclosed herein in a different location that where a trigger mechanism (e.g., a button or lever) is located on the component and/or device.

FIG. 8A is a diagram of a first exemplary optical array 120A. Optical array 120A includes a fixation target display 145, an eye piece lens 830A, a first relay element 835A, a beam splitter 830, a second relay element 835A, an eye/pupil 840, a fixation target path 810, and an optical path to/from a SLO 842. Fixation target display 145 may display a fixation target that emerges from fixation target display as one or more light rays as a first segment 810A of fixation target path 810 that is incident on eye piece lens 820. Eye piece lens 820 may be configured to focus the incident fixation light rays onto a first relay element 835A that emerge from eye piece lens 820 and travel to first relay element 835A via a second segment 810B of fixation target path 810. First relay element 835A may be, for example, one or more optical components (e.g., lenses, mirrors, etc.) configured to direct fixation light rays onto beam splitter 830 via a third segment 810C of fixation target path 810. Beam splitter 830 may be configured to allow transmission of fixation light rays onto second relay element 835B via a fourth segment 810D of fixation target path 810. Second relay element 835B may be configured to focus and/or direct fixation light rays onto the subject eye's pupil 840.

While the fixation target light beams are being projected onto patient's pupil 840, optical scanning of pupil 840 also occurs via scanning radiation (e.g., a laser) projected by a SLO or TSLO along an optical path 842 to/from the SLO/TSLO. The scanning radiation emerges from the SLO/TSLO via a first segment 842A of optical scan path 842 and is incident on beam splitter 830, which is configured to direct the scanning radiation to pupil 840 via a second segment 842B of optical scan path 842. The scanning radiation is reflected by the pupil (or retina) and is incident on beam splitter 830 via second segment 842B of optical scan path 842. Beam splitter 830 then directs the scanning radiation incident on pupil 840 to a detector or the SLO/TSLO via first segment 842A of optical scan path 842.

In some embodiments, optical array 120A may include an eye and/or pupil alignment mechanism 888 configured and arranged to view to facilitate alignment of subject's eye/pupil 840 with optical array 120A by, for example, allowing an operator to view the subject's eye/pupil 840 so that, for example, the alignment of the subject's eye and/or head may be adjusted to align with optical array 120A. Adjusting a position and/or alignment of the subject's head and/or eye/pupil 840 may be facilitated by, for example, adjusting one or more components of ophthalmoscopic device 600, such as head and chin rest 500. Exemplary eye and/or pupil alignment mechanism 888 include, but are not limited to apertures, cameras, lenses that allow a user and/or operator to properly align the subjects eye and/or pupil 840 with optical array 120A.

Some embodiments of the present invention may include a scanning laser ophthalmoscope (SLO) imaging system with monocular and/or binocular imaging optics configured to image a retina of a subject's right and/or left eye of a subject as shown in, for example, FIG. 8B, which is a diagram of a second exemplary optical array 120B that includes fixation path 810 (as seen in FIG. 8A), a scan path 824, and a detection path 832. Scan path 824 includes a plurality of components that direct scanning radiation from an illumination system to the subject's retina and direct scanning radiation reflected from the subject's retina back to a detection system for resolution and processing into one or more retinal images. Scanning radiation directed along scan path 824 is generated an illumination system or scanning radiation source 870 that may emit scanning radiation directed to pupil 840 via a series of components shown in FIG. 8B. In some embodiment illumination system 870 may include an infrared (IR) super luminescent diode (SLD) configured to illuminate the subject's eye and/or retina as IR light emitted therefrom travels along scan path 824 in one or two dimensions (e.g., X, Y, or X and Y directions). Exemplary specifications for a light source coupled to illumination system include use of an infrared light source (e.g., 820-880 nm with a 50 nm bandwidth) via a fixed/continuous output using an optical fiber connection. After exiting illumination system 870, scanning radiation travels to a fiber collimator 872 via scan path 824. Fiber collimator 872 may be arranged and configured to receive the beam of scanning radiation from scanning radiation source 870, collimate the beam of scanning radiation, thereby generating a collimated beam of scanning radiation, and direct the collimated beam of scanning radiation to a first beam splitter 865. First beam splitter 865 may be may be arranged and configured to direct the collimated beam of scanning radiation to a scan path defocus correction assembly 895 that may be arranged and configured to receive the collimated beam of scanning radiation, apply a defocus and/or spherical equivalent correction of a subject's eye or eyes to the collimated beam of scanning radiation, thereby generating a corrected beam of scanning radiation, and direct the corrected beam of scanning radiation through an iris 897 toward a first mirror 845. The scan path defocus correction assembly may include a plurality of components such as a first optical element 860 and a second optical element 855. First optical element 860 and/or second optical element 855 may include a plurality of lenses or other optical components configured to apply a defocus and/or spherical correction to a beam of scanning radiation traveling therethrough. A degree, or feature, of the defocus and/or spherical equivalent correction applied to the collimated beam of scanning radiation may be responsive to imperfections of the subject's eye and/or lens so that, for example, these imperfections to not impact the resolution and/or accuracy of the images of the subject's retina. Additionally, or alternatively, a degree, amount, and/or feature of the defocus or spherical equivalent correction applied to the collimated beam of scanning radiation by the scan path defocus correction assembly may be responsive to an analysis of retinal image quality and may be applied to improve (e.g., reduce blurriness, resolve imaged retinal features with better clarity, etc.) retinal image quality. At times, the defocus or spherical equivalent correction of a subject's eye or eyes applied to the collimated beam of scanning radiation by the scan path defocus correction assembly may be within a range of −12 diopters to +12 diopters. In some embodiments, scan path defocus correction assembly 895 and/or components thereof, may be opto-mechanically controlled.

First mirror 845 may be arranged and configured to direct the corrected beam of scanning radiation to a fast-scanner optical element 867 that may be arranged and configured to receive the corrected beam of scanning radiation and direct the corrected beam of scanning radiation toward a fast scanner optical element 867 that may be arranged and configured to receive the corrected beam of scanning radiation and direct the corrected beam of scanning radiation toward slow-scanning optical element 866. In some embodiments, fast-scanning optical element 867 may be arranged and configured to steer the corrected beam of scanning radiation toward slow-scanning optical element 866 along a first scanning dimension (e.g., along the X-axis). In some embodiments, slow-scanning optical element 866 may be arranged and configured to direct the corrected beam of scanning radiation toward optical element 850 along a second scanning dimension (e.g., along the Y-axis).

Optical element 850 may be arranged and configured to receive the corrected beam of scanning radiation and direct the corrected beam of scanning radiation to a second beam splitter 830 that may be arranged and configured to direct the corrected beam of scanning radiation toward second relay element 835B that may be arranged and configured direct the corrected beam of scanning radiation onto pupil 840 and/or retina, thereby imaging the retina. The scanning radiation may then reflect off of the subject's retina and travel the reverse of the scan path 824 back to first beam splitter 865, which may be directed reflected scan path radiation to a detector assembly along a detection path 832. The detector assembly may include a focusing lens 875 that may be arranged and configured to receive scanning radiation reflected from the subject's retina via first beam splitter 865 and focus the radiation reflected from the subject's retina onto an imaging system 880 that may be arranged and configured to receive scanning radiation reflected from the subject's retina (sometimes referred to herein as detection path data) via first beam splitter 865 and communicate an indication of the scanning radiation reflected from the subject's retina to an external computing device (not shown), such as a processor or cloud computing environment. Imaging system 880 may be, for example, a photodetector and/or an avalanche photo diode (APD) configured to receive and/or measure received scanning radiation and communicate same to, for example, a processor, driver, card, ASIC, and/or FPGA as may be included in, for example, internal computer/processor 135 and/or computer 165. Focusing lens 875 may be configured to achieve optimal retinal focusing on the confocal pinhole and subsequently onto imaging system 880.

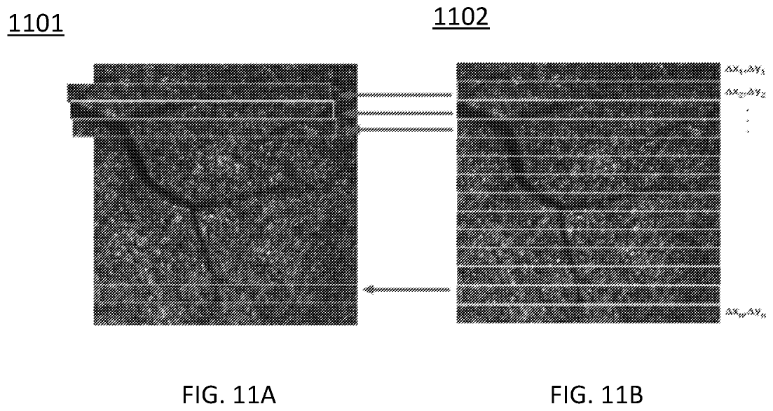

In some embodiments, illumination system 870 may be configured to scan and/or raster scan the retina in the X- and/or Y-dimensions in one direction (e.g., left to right or right to left) and/or two directions (e.g., both left to right and right to left). In some cases, the subject's retina may be raster scanned, pixel by pixel, to subtend a 1-30-degree fOV containing any appropriate number of pixels. Imaging system 880 may be configured to collect back-reflected scanning radiation from the retina and create a high-resolution, motion corrected retinal image, or series of images (e.g., a 1-180 second video) therefrom. Examples of these images are provided in FIGS. 11A, 11B, 12A, and 12C.

In some embodiments, first relay element 835A, aperture 823, and second relay element 835B may cooperate together as a fixation path defocus correction assembly 890 and optical elements 860 and second relay element 855 may cooperate together as a scan path defocus correction assembly 895. In some instances, fixation path defocus correction assembly 890 and scan path defocus correction assembly 895 may be collectively referred to as a “dual defocus correction assembly.” In some embodiments, the components of fixation path defocus correction assembly 890 and/or scan path defocus correction assembly 895 may be opto-mechanically controlled to apply a defocus or spherical equivalent correction of a subject's eye or eyes to the nearest +/−0.25 diopters. Additionally, or alternatively, fixation path defocus correction assembly 890 and scan path defocus correction assembly 895 may be configured to perform simultaneous defocus correction within a range of, for example, −12 diopters to +12 diopters.

In some embodiments, this simultaneous defocus correction may be executed via communicative, electrical, and/or mechanical linking and/or synching of fixation path defocus correction assembly 890 and scan path defocus correction assembly 895 so that, for example, a defocus correction applied to scan path 824 may be scaled and applied to the components of fixation path 810. A degree of defocus correction applied by fixation path defocus correction assembly 890 and/or scan path defocus correction assembly 895 may be determined and/or entered manually by an operator of optical array 120C and/or may be experimentally determined via observing and/or analyzing (by the operator and/or a computer/processor disclosed herein) until the retinal image quality is optimized (e.g., clearest retinal image and/or the highest signal-to-noise ratio (SNR) retinal image). In some embodiments, a degree of defocus correction fixation path defocus correction assembly 890 and scan path defocus correction assembly 895 (or components thereof) may be entered manually and/or automatically responsively to, for example, manual and/or computerized and/or digital analysis of retinal image quality and/or whether an applied correction improves retinal image quality so that, for example, the retinal images with the best and/or highest signal-to-noise ratio (SNR) image are generated.

In some cases, fixation path defocus correction assembly 890 and scan path defocus correction assembly 895 (or components thereof) may be communicatively, mechanically, and/or electrically coupled to one another so that, for example, an adjustment of fixation path defocus correction assembly 890 may trigger a corresponding correction for and/or scan path defocus correction assembly 895 and vice versa. At times, this corresponding correction of the dual defocus assemblies may include automatically scaling and/or focusing light and/or images provided by one or more paths of the SLO system. The synchronization of fixation path defocus correction assembly 890 and scan path defocus correction assembly 895 may be controlled by, for example, internal computer/processor 135.

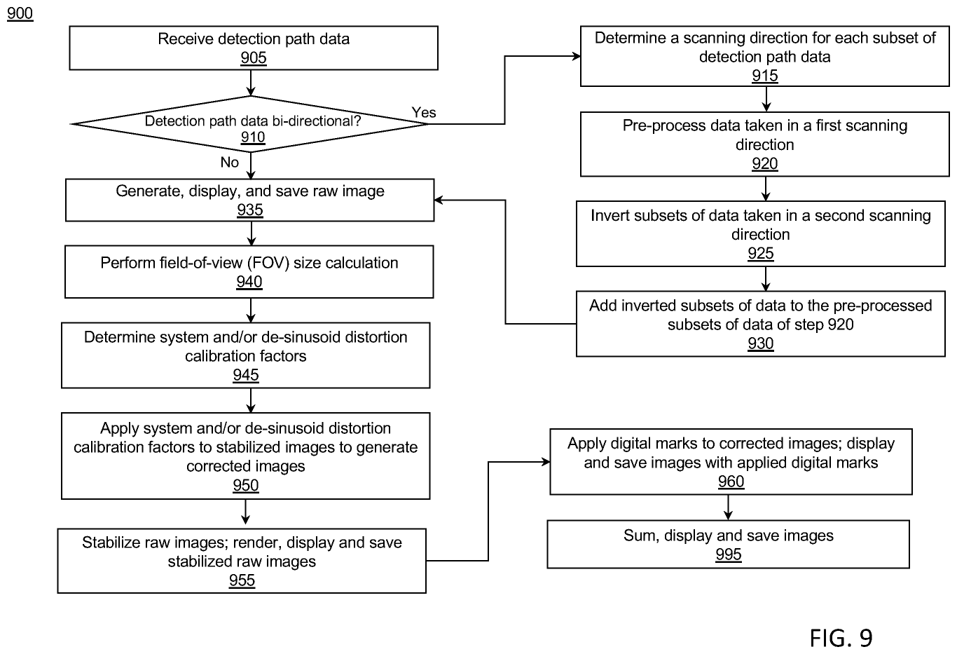

FIG. 9 provides a flowchart of an exemplary process 900 for generating and correcting an image, or a series of images, of a retina using mono-directionally or bi-directionally captured retinal image data. Process 900 may be executed by any of the systems and/or components disclosed herein using, for example, information and/or data received via any of the systems, devices, and/or components disclosed herein. Execution of one or more of the steps of process 900 may be useful and/or necessary in order to, for example, compensate and/or correct for distortion caused by, for example, lenses and other components in the optical array used to generate the detection path data and/or devices used to scan, or otherwise image, the eye/retina.

Initially, detection path data may be received by, for example, imaging system 880 and/or one or more computing/calculation devices (e.g., internal computer/processor 135 and/or computer 165) (step 905). Detection path data may include, for example, retinal imaging data received via an optical array such as optical array 120 and/or a detection path like detection path 832. Often times, the retinal imaging data is received as a series of subsets of detection path data that, in some cases, corresponds to horizontally- or vertically oriented strips that align with a scan path of the device scanning the retina that may be assembled, or stitched together, to generate an image of a portion of the retina corresponding to a field of view via execution of process 900. In some embodiments, the detection path data may include information about one or more components of optical array used to gather the detection path data (e.g., a device used to generate scanning radiation such as illumination system 870) and/or calibration factors and/or corrections that may need to be applied to received detection path data may be known and/or received in step 905. At times, the calibration factors may correct for known flaws or non-linearities of the system generating the detection path data that may be established prior to collection of the detection path data and/or at the time of manufacturing the system. These calibration factors may correct for a variety of conditions such as reflections from one or more lenses, focal distances, known distortions caused by, for example, surface irregularities of one or more lenses, scan fields, beam splitters, and/or mirrors included in the system. Additionally, or alternatively, the calibration factors may be used to synchronize one or more aspects, or subsets, of the detection path data.

In some embodiments, the received detection path data and/or a portion thereof may be pre-processed and/or filtered in order to, for example, remove noise or interference from the data/signal. Exemplary methods of pre-processing the data include, but are not limited to, application of a fast Fourier transform (FFT) to the data, amplifying the data, and/or passing the data through a filter (e.g., a bandpass filter).

In some embodiments, a retina may be sequentially scanned in one, or a first, horizontal direction (e.g., left to right) and, in other embodiments, the retina may be scanned in two, or the first and a second, direction (e.g., from left to right and then from right to left). In step 910, it may be determined whether the detection path data and/or retinal imaging data included therein is mono-directional or bi-directional. When the retina is bi-directionally scanned, a scanning direction for each subset of retinal imaging data may be determined (step 915) and subsets of data taken while scanning in a first direction may be pre-processed for the addition of subsets of data taken while scanning in a second direction (step 920). In step 925, subsets of data taken while scanning in the second direction may be inverted or otherwise processed for the addition of subsets of data taken while scanning in the first direction. At times, execution of one or more pre-processing steps described above with regard to step 905 may be paused when detection path data includes bi-directional data and these pre-processing steps may, instead, be performed during execution of step 925. In step 930, the inverted subsets of data may be added to the pre-processed subsets of data of step 920.

Following execution of step 910 (when the detection path data does not include bi-directional data) or step 930 when the detection path data does include bi-directional data), retinal image data included in the detection path data may then be processed to generate a raw image, which may be rendered, displayed, and saved (step 935). In some embodiments, execution of step 935 may include application of a FFT to some, or all, of the retinal imaging data included in detection path data received in step 905. In step 940, a field of view (FOV) for the detection path data and/or raw image(s) images may be determined. The FOV determination may be made by, for example, determining a number of pixels per degree of retinal scanning data there are and then dividing the total number of pixels by the number of pixels per degree to arrive at the angle (or number of degrees) for the FOV.

In step 945, one or more system and/or de-sinusoidal calibration factors may be determined and/or applied to the stabilized images in order to, for example, remove lens reflections, remove noise from the data, and/or remove non-linearities introduced into the received detection path data by scanning equipment used to generate the detection path data. For example, when illumination system 870 includes a resonance scanner, de-sinusoid distortion calibration factor(s) may be applied to the retinal image to remove the sinusoidal distortions caused by the resonance scanner's oscillation in a sinusoidal pattern. System calibration factors include, but are not limited to, calibration factors to remove reflections from one or more lenses and/or calibration factors to remove irregularities caused by optical instrument flaws or distortions. Some of these calibration factors may be known prior to execution of step 905 and/or may be received during execution of step 905. Additionally, or alternatively, some of these calibration factors may be determined during execution of process 900.

In one embodiment, when a resonance scanner is being used to horizontally scan the retina, determination of a de-sinusoid distortion calibration factors for the retinal image data (step 945) in the form of a horizontal look up table (LUT) may be performed by generating a calibration grid and then, for the horizontal direction, performing, for example, a non-linear least square analysis to solve the sinusoidal equation of the resonant scanner using, for example, calibration grid size, resonant scanner frequency, and pixel clock values. In some cases, calibration grid size, resonant scanner frequency, and/or pixel clock values may be known to the system executing process 900 and/or received in step 905. At times, execution of step 945 may also include generation of a vertical LUT of calibration factors which may be generated by, for example, application of a linear fit analysis across the height of the image and/or a portion of the image. In some embodiments, step 950 and/or a determination of de-sinusoid calibration factors may be based on, or incorporate, the number of pixels per degree and/or the FOV determined in step 940.

In step 950, the system and/or de-sinusoidal calibration factors may be applied to the stabilized image in order to generate corrected images. In some embodiments, execution of step 950 may include applying the calibration factors to the retinal image data and/or stabilized image and then re-rendering the images into linear space by, for example, application of a linear redistribution of the gray scale values across the image. In some embodiments, execution of step 950 may also include determining a fidelity of the correction of the retinal image(s) performed via execution of one or more steps of process 900 by, for example, confirming that the calibration grid appearance in the corrected images is undistorted. In some embodiments, execution of step 950 may incorporate electronic, or digital, subtraction of lens reflections and/or other distortions that may be caused by one or more components of an optical array used to collect the detection path data.

In step 955, image data of step 950 may then be stabilized in order to remove errors and/or noise in the received detection path data caused by, for example, vibrations or other image de-stabilizing causes. In some instances, stabilizing the image may include extracting motion artifacts from the data. Optionally, in step 960, one or more digital marks may be applied to the corrected images and the images with the applied digital marks may be displayed to an operator and saved. Then, in step 965, the images generated and/or corrected via execution of process 900 may be summed, displayed, and saved.

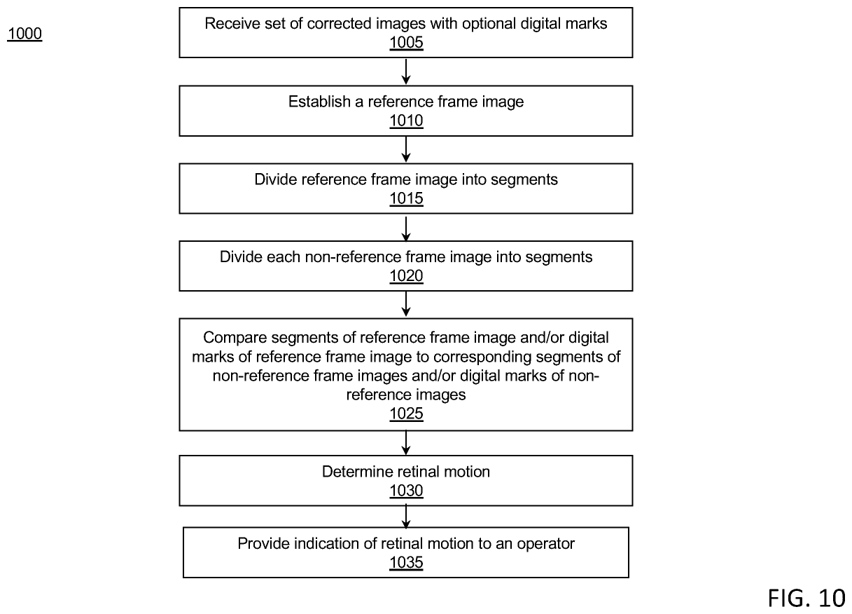

FIG. 10 provides a flowchart of an exemplary process 500 for determining absolute and/or relative movements of the eye and/or retina and providing an indication of same to an operator. Absolute and/or relative movements of the eye/retina may be one or more of a number of horizontal saccade movements and/or microsaccade movements, a number of vertical saccade movements and/or microsaccade movements, an indication of fixational stability, and/or a polar plot of the number of microsaccades. Process 1000 may be executed by any of the systems and/or components disclosed herein using, for example, information and/or data received via any of the systems, devices, and/or components disclosed herein.

In step 1005, a set of corrected images of a retina with optional digital marks may be received. The set of corrected images may be generated by, for example, execution of one or more steps of process 900, described above. In step 1010, a reference frame image for the set of images may be established. In many cases, the reference frame image may be the first image of the set of corrected images. FIG. 6A provides an image 1101 as an exemplary reference frame image as may be established by execution of step 1010. Optionally, in step 1015, the reference frame image may be divided into a plurality (e.g., 12, 24, 28, 30, 40, 100, etc.) of segments, or strips, for further analysis and/or comparison to other images included in the set. In some cases, the segments may be horizontally oriented strips. In some embodiments, the reference frame image and/or non-reference frame images may be overlapping by, for example, 1-25 pixels.

In step 1020, the non-reference frame images may be divided into a number (e.g., 8, 14, 16, 32, etc.) segments, or strips. Often times, when step 1015 is performed, the number of segments of the reference frame image and the non-reference frame images may be the same. Next, the reference frame as a whole (e.g., when step 1015 is not executed) and/or segments of the reference frame image (e.g., when step 1015 is executed) and/or digital marks of the reference frame image may be compared with corresponding segments of each non-reference frame image and or digital marks on each non-reference frame image to determine differences therebetween (step 1025). FIG. 11B provides an exemplary non-reference frame image 1102 from the set received in step 1005 divided into fourteen segments and shows how the second, third, and fourth segments of non-reference frame image 1102 must be moved to the left (as shown in FIG. 6A) to align with corresponding portions and/or segments of the reference image.

In step 1030, a degree of retinal motion (e.g., a change is position, a velocity of movement, and/or a speed of movement) may be determined using comparison results from execution of step 1025 and an indication of the retinal motion may be provided to an operator (step 1035). In the case of FIGS. 11A and 11B, the retinal motion may be determined by calculating a difference in the position (in the X- and/or Y-dimensions) of a feature in non-reference frame image when compared with the reference frame image. This difference in position, along with a measure of time between the capture of the reference frame image and the non-reference frame image may be used to calculate a velocity, direction, or rate, of retinal movement.

In one exemplary embodiment, process 500 may be performed to determine X- and Y-dimension displacements that may be reported up to, for example, 32 times per image at a reporting rate of 960 Hz. In some embodiments, process 500 may be performed in situ (i.e., while the subject is using system 100 and/or a component thereof) in real-time so that the operator can see both the subject's actual retinal motion and the stabilized version of the retina side by side on a software interface displayed to the operator.

FIGS. 12A, 12B, 12C, and 12D provide exemplary graphic user interfaces (GUI) and window 1200, 1201, 1202, 1203, and 1204, respectively, that are four examples of how an indication of retinal motion along with other information may be provided to an operator via execution of step 1035 or otherwise. GUI 1200 of FIG. 12A includes a menu bar 1210 that provides information about the subject (e.g., name, date of scan, etc.) along with a plurality of options for displaying information about the subject's retina. Options for displaying information about the subject's retina provided by menu bar 1210 include an option of displaying fixation stability metrics, fixation stability video, target tracking metrics, and target tracking video. Additionally, or alternatively, information regarding saccades, fixation, and/or smooth pursuit movements of a retina may also be displayed and/or available. A first window 1201 provides a GUI displaying information pertaining to fixation stability metrics and a second window 1202 providing pertaining to a fixation stability video.

FIG. 12B provides a close-up view of first window 1201, which displays information pertaining to retinal fixation stability metrics. In particular, first window 1201 and provides a polar plot of fixation stability 715, a polar plot of microsaccade amplitude 720, a table of average fixation metrics 725, a graph of vertical motion 730 that plots vertical retinal motion amplitude in degrees as a function of time in seconds, and a graph of horizontal motion 735 that plots horizontal retinal motion amplitude in degrees as a function of time in seconds as shown.

FIG. 12C provides a close-up view of second window 1202, which displays information pertaining to retinal fixation stability videos. Second window 703 provides a polar plot of fixation stability 1245, a polar plot of microsaccade amplitude and direction 1250, and a window 1255 showing the reference frame from a video of retinal movement along with a set of GUI controls for displaying the video.

FIG. 12D includes menu bar 1210 that indicates the operator has selected for the display of target tracking metrics. GUI 704 also includes a third window 703 and a fourth window 704, both of which provide information regarding horizontal speed and tracking and vertical speed and tracking as shown.

FIGS. 13A-13D provide diagrams of a fixation target display such as fixation target display 145 displaying a first fixation target image 1301, a second fixation target image 1302, a third fixation target image 1303, and a fourth fixation target image 1304, respectively, that may be provided to a subject via, for example, a fixation target path such as fixation target path 810. First fixation target image 1301 includes a single black crosshair positioned in an approximate center of image 1301. Second fixation target image 1302 includes two horizontally aligned black crosshairs positioned approximately 5 degrees apart. Third fixation target image 1303 includes two vertically aligned black crosshairs positioned approximately 5 degrees apart. First image 1301 may be used to perform a fixational motion measurement of the subject's eye(s); second image 1302 may be used by the subject to perform a voluntary horizontal saccade motion measurement of the subject's eye(s); and third image 1303 may be used by the subject to perform a voluntary vertical saccade motion measurement of the subject's eye(s).

Fourth fixation target image 1304 includes 9 crosshairs arranged in three row and columns of three crosshairs each. The crosshairs in the horizontal rows may be positioned approximately 5 degrees apart. In some embodiments, the nine fixation targets of fixation target image 1304 may be used to direct a subject to look at each fixation target in turn (e.g., first, second, third, fourth, etc.) or in various combinations (e.g., first, third, fifth, seventh, and ninth; or third, fifth, seventh, first, fifth, and ninth; or second, fifth, eighth, fourth, fifth, sixth) to capture a plurality (e.g., 2-20) individual, or unique 5-degree fOV retinal images as the subject voluntarily focuses on fixation targets.

In some cases, two or more of the fixation targets of second, third, and/or fourth fixation target image(s) 1302, 1303, and/or 1304 may be positioned further apart (e.g., 5.2-15 degrees) and/or closer together (e.g., 4.9-0.1 degrees) in order to direct the subject to voluntarily focus on fixation targets that are closer together or further apart to facilitate the capturing of a plurality of retinal images (e.g., a series of individual images taken over time and/or a video) with different fields of view. For example, if fixation targets are positioned close together, or overlap (e.g., a 0.1-0.5 degree fOV overlap), corresponding retinal images captured when the subject voluntarily focuses on the different fixation targets may have overlapping subject matter (e.g., two or more images may capture the same area of the retina, particularly along an edge of the image corresponding to the overlap of the fixation targets). This overlapping subject matter may facilitate alignment of a plurality of corresponding retinal images when using them to construct a composite high-resolution retinal image that shows a larger surface area of the retina than an individual retinal image that has, for example, a 5-30 degree fOV. In some embodiments, this composite image may serve as a reference frame for the subject's retina that may be used to, for example, find and/or determine a position for one or more features of the subject's retina as shown in individual images of the subject's retina within the larger FOV shown in the composite image or as otherwise described herein. For example, a composite retinal image may be used as a reference frame in, for example, step 1010 of process 1000 described above.

In some embodiments, use of a composite image as a reference frame in this way may aid in the processing of individual images to, for example, determine changes in position of the retina (via, for example, measuring changes in position of retinal features) over time, which may assist in the rapid processing and analysis of a plurality of retinal images to determine characteristics thereof. Additionally, or alternatively, the high resolution, larger FOV reference frame, composite retinal image may allow for the imaging and/or analysis of larger and/or faster eye movements, versus using a single retinal image frame with, for example, a 5 degree fOV, as the eye-tracking reference. Additionally, or alternatively, using a composite retinal image in this way may allow for the capture and analysis of retinal images to determine, for example, fixation instability, as well as attributes (e.g., direction, velocity, amplitude, etc.) of voluntary saccades and/or involuntary eye movements (e.g., microsaccades) in the horizontal and/or vertical directions because, for example, a cross-correlation threshold required for each strip or segment of non-reference frame images (0.8 or 80%) may be met with the composite retinal image when it otherwise would not be.

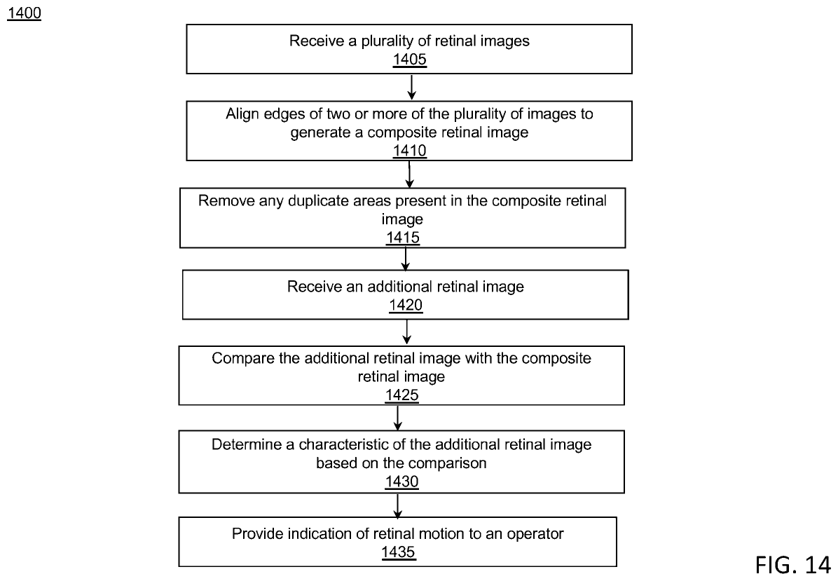

FIG. 14 provides a flowchart of an exemplary process 1400 for generating a composite retinal image for a subject's retina and using the composite retinal image to determine a feature or characteristic of a subsequently received image of the subject's retina. Process 1400 may be executed by any of the systems and/or components disclosed herein using, for example, information and/or data received via any of the systems, devices, and/or components disclosed herein.

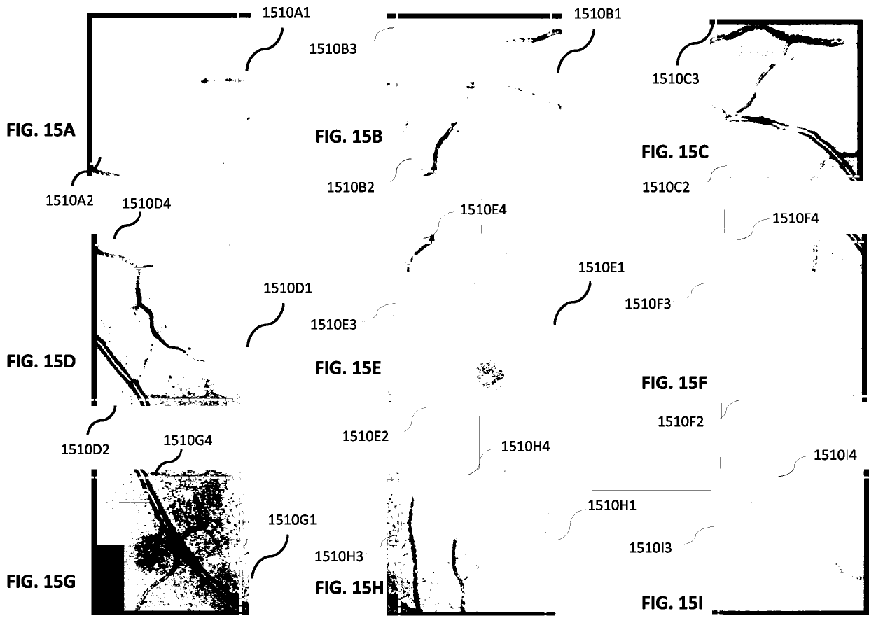

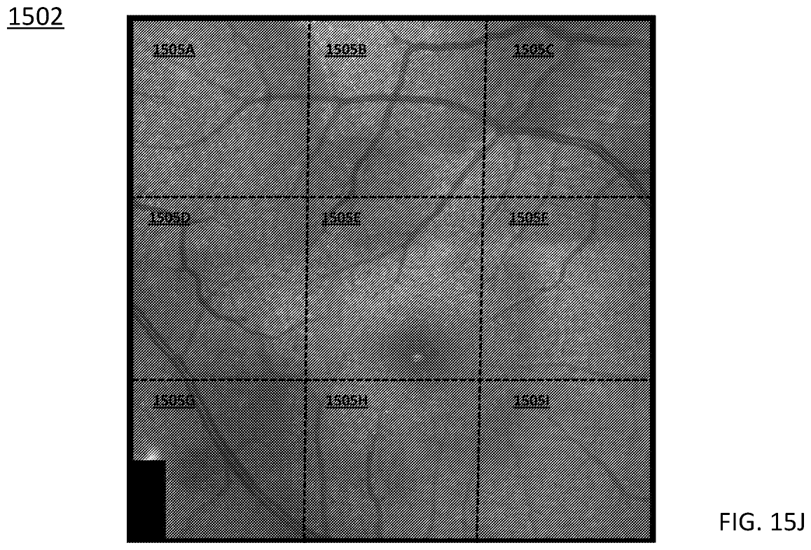

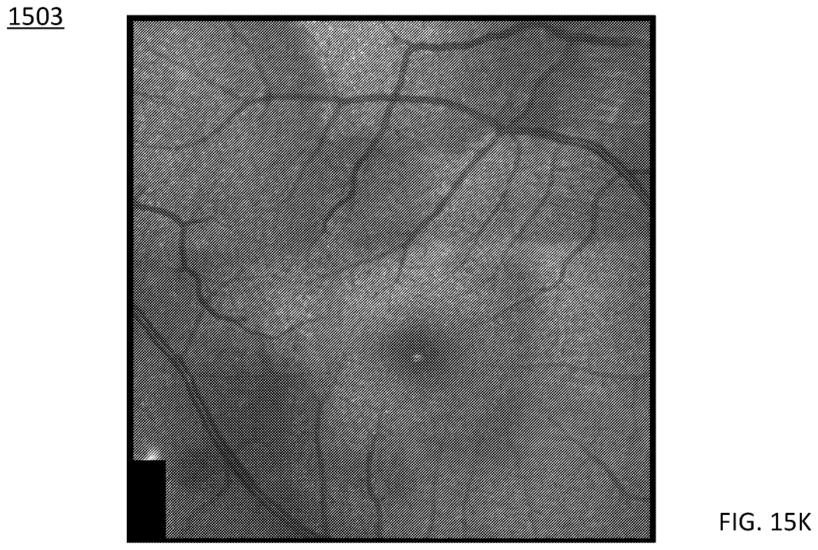

Initially, in step 1405, a plurality (2-70) of images of a subject's retina may be received by, for example, a processor or computer such as internal computer/processor 135, computer 165, and/or machine learning/deep neural network architecture 180. The retinal images may be received as, for example, detection path data received via an SLO like detection path data 832 and rendered into images by the receiving processor or computer. In some cases, each image of the plurality may image a different region, or field of view, of the subject's retina. At times, a portion of subject matter (e.g., retinal field of view) of one image of the plurality may be the same as and/or overlap with the subject matter of another image. This may occur when, for example, the subject's retina is imaged with overlapping fields of view. An exemplary set of retinal images that may be received via execution of step 1405 is provided by FIGS. 15A-15I.

In step 1410 edges of two or more of the plurality of images may be aligned with one another to form a composite retinal image, such as composite retinal image 1502 or 1503 as shown in FIGS. 15J and 15K, respectively. For example, when forming the composite retinal image, a left edge of a first image of the plurality may be aligned with a right edge of a second image of the plurality and aligned so that a feature (e.g., blood vessel) that is shown in both the first and second images is properly aligned within a composite of the first and second image. Optionally, formation of the composite retinal image may include removing any duplicate information (e.g., a portion of two aligned images of the plurality show the same area of the subject's retina) (step 1415).

In step 1420, an additional image of the subject's retina may be received. The additional retinal image received in step 1420 may have been taken close in time (e.g., minutes or hours) to when the plurality of images received in step 1405 or at a later time (e.g., months or years). The additional image may be compared with the composite retinal image of, for example, 1415 or 1420 (step 1425) and a characteristic of the additional retinal image and/or the retina may be determined based on the comparison (step 1430). In some embodiments, the composite retinal image of step 1415 and/or 1420 may be used as a reference image and may be used as such in the processes (e.g., process 1000, 1400, and/or 2200) as disclosed herein.

FIGS. 15A-15I are a plurality of images of a subject's retina from different fields of view that may be arranged and combined to form a composite image of the subject's retina with a larger field of view than the field of view provided by any single image. FIGS. 15A-15I are arranged in a grid-like manner with three rows of images arranged in three columns. The fields of view for each of first-ninth images 1505A-1505I may be, for example, three-ten degrees. In particular, FIG. 15A provides a first image 1505A that shows an upper-left field of view of the subject's retina with a right-side overlapping region 1510A1 along a right edge of first image 1505A and a lower overlapping region 1510A2 along a lower edge thereof. FIG. 15B provides a second image 1505B that shows an upper-center field of view of the subject's retina with a right-side overlapping region 1510B1 along a right edge of second image 1505B, a lower overlapping region 1510A2 along a lower edge, and a left-side overlapping region 1510B3 along a left edge thereof. FIG. 15C provides a third image 1505C that shows an upper-right field of view of the subject's retina with a lower overlapping region 1510C2 along a lower edge and a left-side overlapping region 1510C3 along a left edge thereof.

FIG. 15D provides a fourth image 1505D that shows a center-left field of view of the subject's retina with a right-side overlapping region 1510D1 along a right edge of fourth image 1505D, a lower overlapping region 1510A2 along a lower edge, and an upper overlapping region 1510D4 along an upper edge thereof. FIG. 15E provides a fifth image 1505E that shows a center field of view of the subject's retina with a right-side overlapping region 1510E1 along a right edge of fifth image 1505B, a lower overlapping region 1510E2 along a lower edge, a left-side overlapping region 1510E3 along a left edge, and an upper overlapping region 1510E4 along an upper edge thereof. FIG. 15F provides a sixth image 1505F that shows a center-right field of view of the subject's retina with a lower overlapping region 1510F2 along a lower edge, and a left-side overlapping region 1510F3 along a left edge, and an upper overlapping region 1510F4 along an upper edge thereof.

FIG. 15G provides a seventh image 1505G that shows a lower-left field of view of the subject's retina with a right-side overlapping region 1510G1 along a right edge of seventh image 1505G and an upper overlapping region 1510G4 along an upper edge thereof. FIG. 15H provides an eighth image 1505H that shows a lower-center field of view of the subject's retina with a right-side overlapping region 1510H1 along a right edge of seventh image 1505B, a left-side overlapping region 1510H3 along a left edge, and an upper overlapping region 1510H4 along an upper edge thereof. FIG. 15I provides a ninth image 1505I that shows a lower-right field of view of the subject's retina with an upper overlapping region 1510I4 along an upper edge and a left-side overlapping region 1510I3 along a left edge thereof.

FIG. 15K provides a photograph of an exemplary composite retinal image 1501 that may have been generated via execution of process 1400 using the plurality of retinal images 1505A-1505I of FIGS. 15A-15I, respectively. On some occasions, retinal image 1501 may be a reference frame and may be used as such as, for example, disclosed herein. In some embodiments, composite retinal image 1501 may be generated by assembling nine images with varying fields of view into composite retinal image 1501. The plurality of retinal images used to generate composite retinal image 1501 may have been taken when, for example, the subject voluntarily focused on each of 9 fixation targets provided by, for example, fourth fixation target image 1304. In some embodiments, generation of composite retinal image 1401, may be facilitated by matching and/or aligning the nine unique retinal images with one another via an overlapping portion, or edge, of each of the individual images. For example, first retinal image 1505A may be combined with second retinal image 1505B by aligning right-side overlapping region 1510A1 with left side overlapping region 1510B3 of second retinal image 1505B as shown in FIG. 15J so that no duplicate portions of the retina are shown in composite retinal image 1502. This process may be repeated (e.g., alignment and removal of duplicative material) for all nine images used to generate composite retinal image 1502 by aligning each of first-ninth retinal images 1505A-1505I along the gridlines shown in FIG. 15J.

On some occasions, the high resolution, larger FOV reference frame provided by composite retinal image 1502 may provide a larger reference frame with which to compare non-reference frame images (e.g., process 1000, 1400, etc.), which increases a likelihood that most, or all, content of non-reference frame images may be aligned with composite retinal image 1401 (when acting as the reference frame image). This may allow for the measurement of larger and faster eye movements (embodied as non-reference frames capture portions of the retina not shown in a non-composite retinal image reference frame). This can allow for the accurate capture of fixation instability, drift, and/or voluntary saccades in both the horizontal and vertical directions that may have either too high a velocity and/or amplitude value to be accurately measured using a single retinal image frame of reference because, for example, a cross-correlation threshold required for each strip or segment of non-reference frame images (0.8 or 80%) may be met with composite retinal image 1401 when it otherwise would not be. This allows for the accurate measuring of a high velocity and/or high amplitude eye movements and may improve the overall accuracy of analysis of retinal images taken with and/or analyzed via the systems and processes disclosed herein.

FIG. 15K shows the composite retinal image of FIG. 15K without the two horizontal and two vertical gridlines (of FIG. 15J) superimposed thereon.

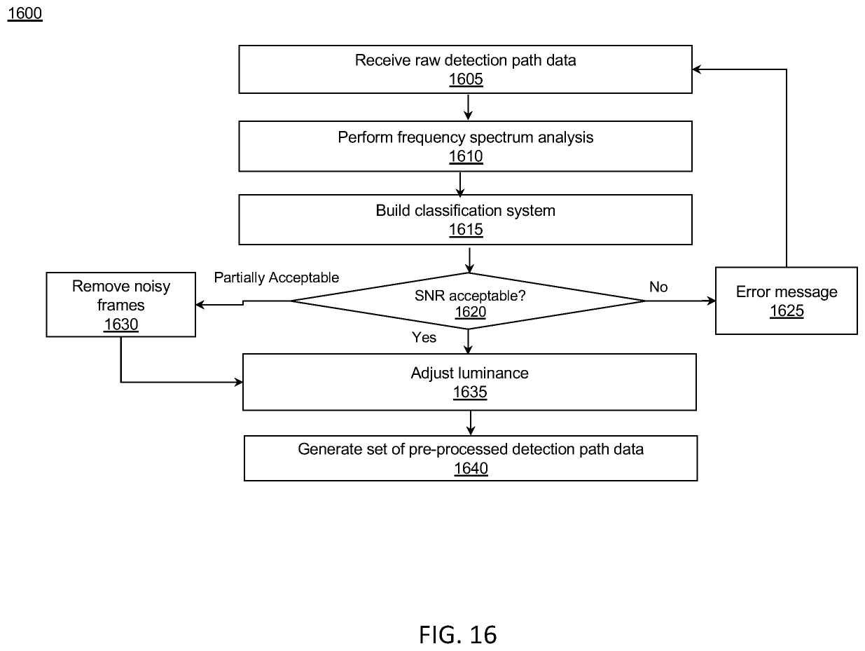

FIG. 16 provides a flowchart of an exemplary process 1600 for evaluating a strength and/or quality of a detection path signal (that may be used to generate a retinal image as described herein), or portions thereof (e.g., images and/or frames that include image data) captured using a SLO and/or generating a set of pre-processed detection path data. The detection path data may include imaging data for a subject's retina in the form of one or more frames of a set of frames (i.e., video). Process 1600 may be executed by any of the systems and/or components disclosed herein using, for example, information and/or data received via any of the systems, devices, and/or components disclosed herein.

In some cases, execution of one or more of the steps of process 1600 may be useful and/or necessary in order to, for example, remove a portion (e.g., a frame or set of frames) of a detection path data signal (e.g., a video) that is not of sufficient quality (e.g., SNR below a threshold value and/or brightness that does not allow for distinguishing anatomical features of the retina) to be further analyzed via execution of, for example, one or more processes disclosed herein. Removal of noisy and/or low quality images and/or frames from detection path data may lead to more efficient and/or higher accuracy analysis of detection path data and/or retinal images generated therefrom because, for example, the detection path data and/or retinal images generated therefrom that is analyzed may not be polluted with low-resolution and/or noisy data and/or image frames.

Initially in process 1600, a set of raw detection path data may be received by, for example, imaging system 880 and/or one or more computing/calculation devices (e.g., internal computer/processor 135 and/or computer 165) (step 1605). The raw detection path data may be data received via a detection path such as detection path 832 shown in FIG. 8B and may include one or more images of a subject's retina that has been imaged via, for example, a retinal scanning path such as scan path 810. In some embodiments, the raw detection path data may correspond to a set of images and/or a video of the retina that includes a plurality (e.g., 30, 60, 90, 180) frames or retinal images taken over a period of time (e.g., 5-240 seconds).

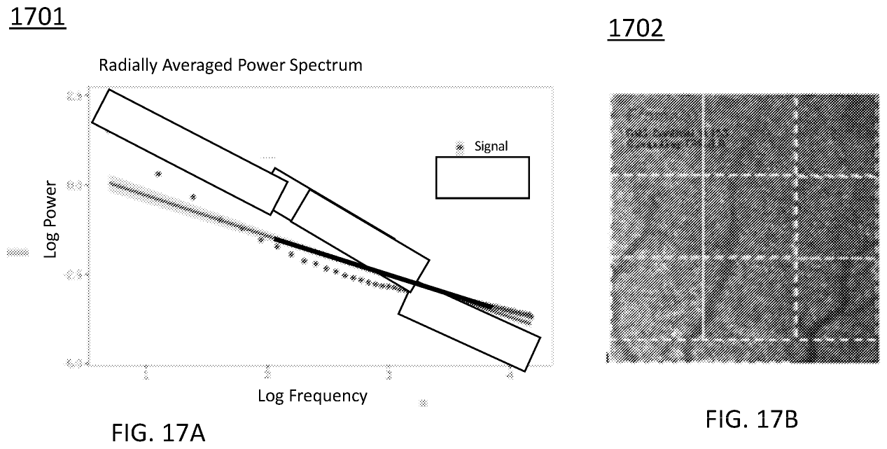

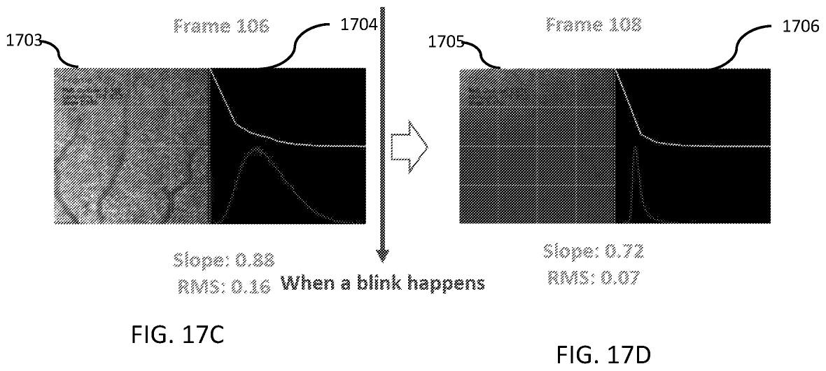

In step 1610 a frequency spectrum analysis may be performed on the received raw detection path data and/or a subset of the raw detection path data that may correspond to one or more images and/or frames of the subject's retina. In some cases, execution of step 1610 may include application of a GPU accelerated fast Fourier transform (FFT) to the raw detection path data in order to, for example, analyze a radially distributed frequency power spectrum for each frame. A result of the frequency spectrum analysis of step 1610 may be a graph, such as graph 1701, showing a radially averaged power spectrum for subset of raw detection path data corresponding to an image 1702 shown in FIG. 17B. Generation of graph 1701 includes plotting a log of the frequency (X-axis) of the raw image data corresponding to image 1702 as a function of a log of the power (Y-axis) of the raw image data corresponding to image 1702 and then performing a linear regression to determine a slope of the plotted data points. Power, in this analysis, may be a square of magnitude of the amplitude of the signal, or signal amplitude squared.

Optionally, a classification system for each of the images and/or frames included a set of images, or video, included in the raw detection path data may be built and/or determined (step 1615). The classification system built in step 1615, may, for example, classify subsets of raw detection path data that may correspond to a single, or set of images, that may be generating using the raw detection path data using, for example, a magnitude or feature of one or more characteristics (e.g., power, frequency, intensity, etc.) of the raw detection path data and/or relative relationship(s) between the characteristics. For example, in some embodiments, the classification system may be determined using, for example, a linear regression calculation to compute an optimal slope value, or optimal range of slope values, of the frequency distribution determined in step 1610, wherein slopes above the optimal value and/or within an optimal range of values indicate that an image, or set of images, within the raw detection path data that has an acceptable signal-to-noise ratio.

In step 1620, a signal-to-noise ratio (SNR) for a portion of the raw detection path data (e.g., one or more images and/or frames) may be determined and compared with a threshold SNR value to determine whether the SNR for the portion of the raw data (e.g., the one or more images(s)) is acceptable (e.g., above the threshold). In some instances, the threshold SNR may be embodied as a slope of a radially distributed frequency power spectrum for an image or frame of detection path data (as determined in step 1610) and execution of step 1620 may include determining a slope of one or more images and/or frames as described above with regard to FIGS. 17A and 17B, to determine whether a slope of a plot of a log of the frequency as a function of a log of the power for the one or more subsets of raw data (e.g., images and/or frames) are above an optimal value. Additionally, or alternatively, execution of step 1620 may include determining an average slope of all and/or a subset of the raw detection path data and/or images and/or frames corresponding to all, or a subset of, the raw detection path data based on, for example, individual calculations for each of the images and/or frames included within the raw detection path data and/or a subset thereof.

When the SNR is below the threshold value or otherwise not acceptable (step 1620), an error message may be communicated to an operator of the system that generated the raw detection path data (e.g., optical measurement device 105) (step 1625) and, at times, step 1605 may be executed again with a new set of raw detection path data.

In some cases, process 1600 may be done in real time, or near real time (e.g., a 1-15 minute lag time) so that an operator can receive feedback regarding raw detection path data quality and/or SNR so that the operator may determine whether, for example, the subject's retina needs to be rescanned, an adjust to the equipment used to scan the subject's retina is required, and/or a process used to generate the raw detection path data needs to be modified and/or repeated. In some embodiments, a subset of raw detection path data (e.g., images and/or frames within a video) may be too noisy (e.g., when the subject blinks) and a remainder of the subset of raw detection path data may have an acceptable SNR (step 1620). When this happens, the noisy subsets of raw detection path data (e.g., frame(s) and/or image(s) that correspond to the subject's blinks) may be removed from the raw detection path data (step 1630) and the remainder of the now edited detection path data may be further analyzed and/or processed to, for example, determine characteristics of retinal motion, according to one or more processes disclosed herein.

Optionally, when the SNR of the edited detection path data is acceptable (step 1620) the edited detection path data and/or image/frames included therein may be further processed (step 1635) to, for example, make analysis thereof easier. Exemplary processing that may occur during execution of step 1635 includes, but is not limited to, adjust a luminance of one or more images generated using the edited detection path data so the luminance is relatively and/or approximately consistent over the set of images and/or video. In some embodiments, step 1635 may be executed by, for example, application of a Gaussian blur image filter to smooth photon distributions collected within each frame and/or image.

FIGS. 17C and 17D provide one example of how steps 1605-1635 may be performed. FIGS. 17C and 17D provide a first and a second retinal image 1703 and 1705, respectively, that were generated using raw detection path data associated with frame 106 and frame 108, respectively, of a set of raw detection path data received in step 1605. FIGS. 17C and 17D further provide a first graph 1704 that plots mathematical analysis of a slope (0.88) and a root mean square (RMS) (0.16) of first image 1703 and a second graph 1706 that plots mathematical analysis of a slope (0.72) and an RMS (0.07) of second image 1705. First and second graphs 1704 and 1706 may be generated via, for example, execution of step 1610. First image 1703 shows anatomical features (e.g., capillaries and cells) of a subject's retina, which may be distinguished from one another via, for example, observation of relatively light and dark regions of first image 1703. Second image 1705 is mostly dark and does not show any anatomical features of the retina. Mathematical analysis of first image 1703 in the form of radially averaged power spectrum yields an average slope of 0.88 for the image and also provides an RMS value of 0.16. In the embodiment of this example, an average slope value below a threshold of 0.75 may indicate that the image frame is too noisy for further analysis (i.e., the SNR is not acceptable) in step 1620. Since the average slope value for first image 1703 is above this threshold, it may be deemed to have acceptable SNR in step 1620. In contrast, second image 1705 may be considered noise, or to have an unacceptable SNR, because the slope for second image 1705 is 0.72, which is below the 0.75 SNR threshold. In addition, the RMS for second image 1705 is 0.07. Because of the low slope and RMS value, the image of frame 108 may be considered noise as may occur when, for example, the subject blinks and, in some cases, frame 108 may be removed from the set of images/frames via execution of step 1630 when the edited detection path data is created.

In step 1640, a set of pre-processed detection path data may be generated that includes, for example, the raw detection path data received in step 1605, the edited detection path data generated via execution of step 1630, and/or the set of images and/or frames with adjusted luminance generated via execution of step 1635. The preprocessing may be performed to, for example, make analysis of the retinal images easier and/or more efficient. Exemplary types of pre-processing that may be performed via execution of step 1640 include, but are not limited to, filtering (e.g., bandpass filtering) the data of step 1605, 1630, and/or 1635 and/or applying a noise reduction algorithm to the data of step 1605, 1630, and/or 1635. Additionally, or alternatively, processing that may occur during execution of step 1640 may include amplification and/or contrast adjustment of one or aspects of one or more images generated using the edited detection path data.

FIG. 18 provides a flowchart of an exemplary process 1800 for training, for example, a machine learning architecture to recognize features of a retinal image. Process 1800 may be executed by, for example, any of the systems and/or components disclosed herein using, for example, information and/or data received via any of the systems, devices, and/or components disclosed herein. In some embodiments, a machine learning and/or deep neural network executing process 1800 may be generative adversarial network (GAN) based learning framework.

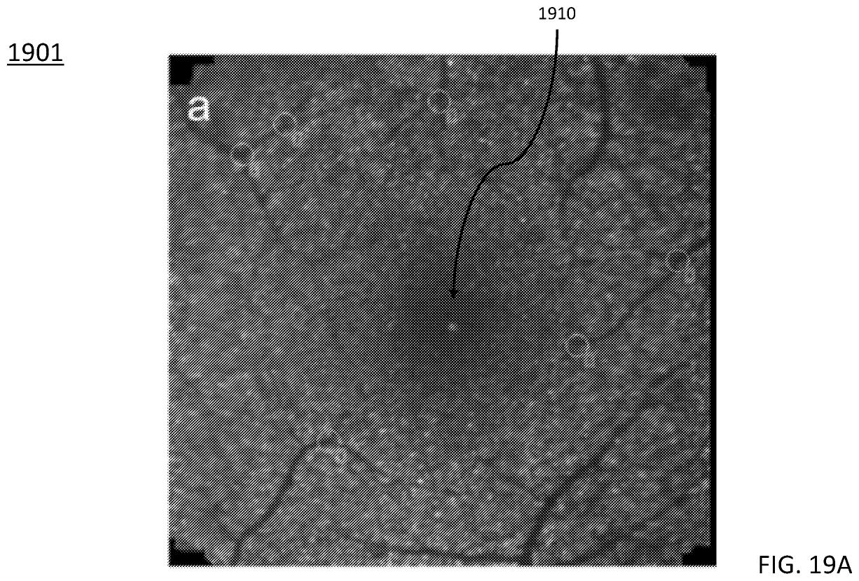

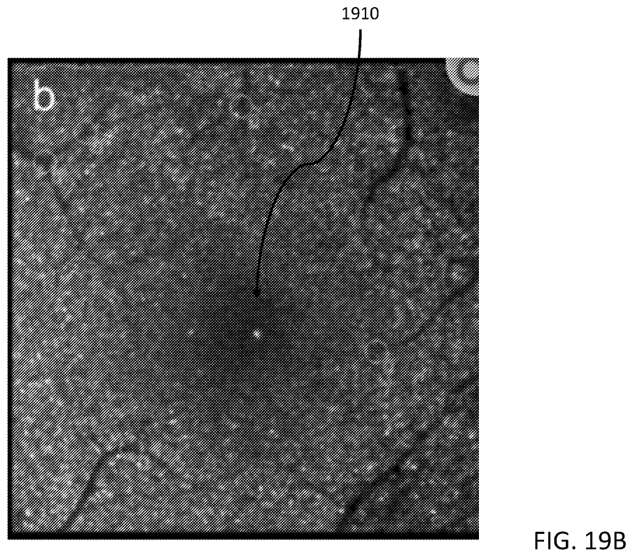

In step 1805 a set of marked retinal images may be received. The retinal images may be marked to point out various features, such as anatomical features present thereon. Exemplary anatomical features that may be marked on one or more retinal images include, but are not limited to, fovea, macula, capillaries, capillary branches, vasculature, vascular branches, hemorrhages, exudates, retinal abnormalities, retinal anomalies, injuries, and/or retinal photoreceptors. The anatomical features may present themselves in the retinal images as regions of varying light intensity levels (e.g., greyscale) of varying shapes and patterns. For example, blood vessels and capillary networks have unique shapes that show as dark-colored (light-absorbing) vessels crossing in the image. At times, the retinal images marked by a human who analyzes the retinal images. Additionally, or alternatively, in some embodiments, the marked retinal images received in step 1805 may be generated via, for example, execution of process 900 and, in particular, step 960. FIGS. 19A and 19B are photographs of a first exemplary retinal image 1101 and a second exemplary retinal image 1902 with markings of various features superimposed thereon like the retinal images that may be received in step 1805. In the example of images 1901 and 1901, the retina's fovea 1910 is marked with an arrow and capillary branches are marked with green circles.

In step 1810, the set of marked retinal images received in step 1805 may be divided into a training set of marked retinal images and a test set of marked retinal images. The training set of marked retinal images may include 60-90% of the marked retinal images and the test set of marked retinal images may include the remainder of marked retinal images not included in training set of marked retinal images (i.e., the remaining 10-40%). Machine learning and/or deep neural network computer architecture inputs may be selected and/or set up (step 1815) for entry into a machine learning and/or deep neural network computer architecture like machine learning and/or deep neural network computer architecture 180. These inputs may include instructions for how to analyze and/or categorize the training and/or test set of marked retinal images, instructions for detecting a marking on a retinal image, instructions for detecting a determining a characteristic (e.g., size, position, orientation, etc.) of a feature marked on a retinal image, instructions for recognizing different types of features in the marked retinal images, instructions for differentiating between different types of features included in the marked retinal images, and/or instructions for generating an output (e.g., an algorithm and/or model) of the machine learning and/or deep neural network analysis. In some cases, the machine learning and/or deep neural network architecture inputs may be specific to the type of machine learning and/or deep neural network architecture being used.

In step 1820, the training set of marked retinal images may be run through, or otherwise processed by, the machine learning and/or deep neural network architecture to generate a first, or primary, version of a retinal feature detection model and/or algorithm, which may be configured and/or optimized to receive unmarked retinal images and recognize features therein.

In step 1825, the first version of the retinal feature detection model and/or algorithm may be tested using the testing set of marked retinal images to determine, for example, a level of accuracy of the first version of the retinal feature detection model and/or algorithm. Results of the testing may be evaluated (step 1830) and results of the evaluation may be used to update or iterate the first version of the retinal feature detection model and/or algorithm thereby generating a second version of the retinal feature detection model and/or algorithm (step 1835). In some embodiments, one or more steps of process 1800 (e.g., steps 1820-1830) may be repeated until the second (or subsequent) version of the retinal feature detection model and/or algorithm is configured to detect features of retinal images with an acceptable level of accuracy, precision, and/or confidence.

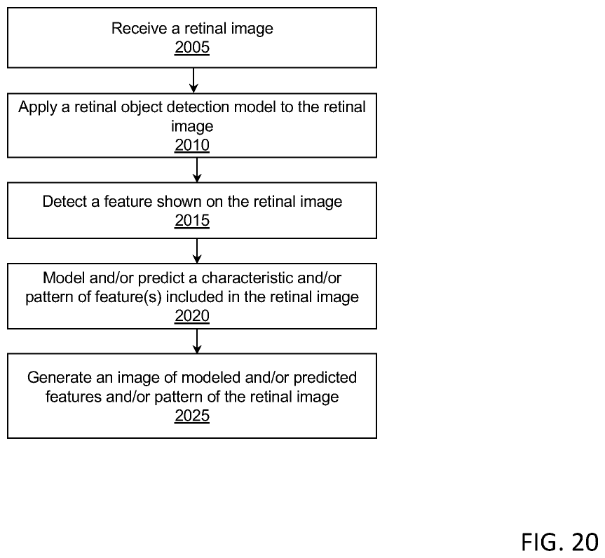

FIG. 20 provides a flowchart of an exemplary process 2000 for predicting and/or modeling features of a retinal image. Process 2000 may be executed by, for example, any of the systems and/or components disclosed herein using, for example, information and/or data received via any of the systems, devices, and/or components disclosed herein.

In step 2005, a retinal image, such as the retinal images and/or sets of retinal images described herein may be received. In some embodiments, the retinal images received may correspond to edited and/or preprocessed detection path data/images as generated and discussed above with regard to process 1600. Often times, the retinal images received in step 2005 will not be marked to show a position of features of interest. In step 2010, a retinal feature detection model and/or algorithm, such as the second version of the retinal feature detection model and/or algorithm generated via execution of process 1800, may be applied to the retinal image(s) received in step 2005 to detect a feature of the retina shown or otherwise provided by the retinal image(s) (step 2015). Exemplary features of the retina include, but are not limited to, fovea, macula, capillaries, capillary branches, vasculature, vascular branches, hemorrhages, exudates, retinal abnormalities, retinal anomalies, injuries, and/or retinal photoreceptors.

In step 2020, one or more characteristics of features included in the retina may be modeled and/or predicted. In some cases, execution of step 2020 may include predicting and/or modeling how different features (e.g., capillary branches) detected in step 2015 fit together, or form a pattern, within the retinal image. In some embodiments, execution of step 2020 may include reducing a number of details provided by the retinal images so that, for example, only a few features (e.g., blood vessels) are modeled and/or predicted. A purpose of executing step 2020 is to reduce the complexity of analyzed images to, for example, reduce processing time for analyzing the image. In step 2025, an image of the modeled and/or predicted features of the retina may be generated.

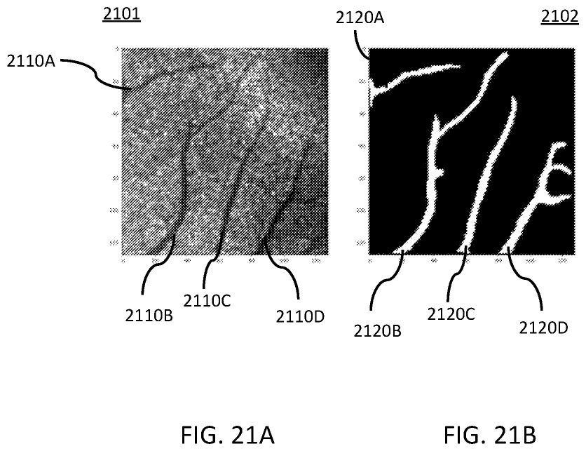

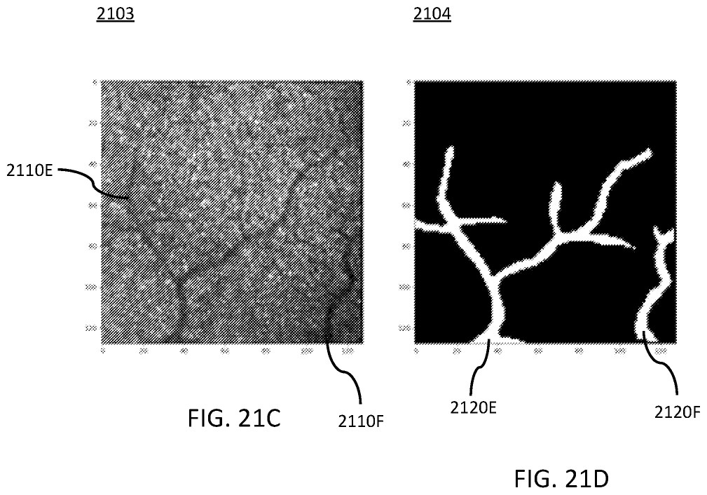

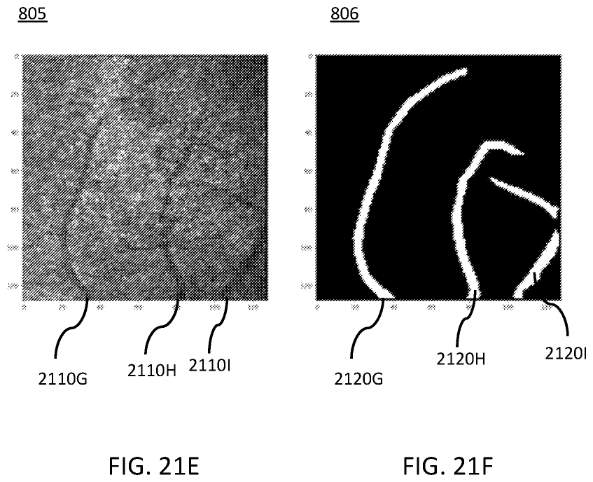

FIGS. 21A-21F provide an example of how process 2000 may be executed, wherein FIGS. 21A, 21C, and 21E provide a first, second, and third retinal image 2101, 2103, and 2105, respectively that may be received in step 2005 and FIGS. 21B, 21D, and 21F provide a corresponding first, second, and third image 2102, 2104, and 2106, respectively of modeled features of the retina shown in first, second, and third retinal image 2101, 2103, and 2105, respectively. In particular, first retinal image 2101 shows an actual first blood vessel 2110A, an actual second blood vessel 2110B, an actual third blood vessel 2110C, and an actual fourth blood vessel 2110D and image of a model of first retinal image 2102 shows a modeled first blood vessel 2120A, a modeled second blood vessel 2120B, a third blood modeled vessel 2120C, and a fourth modeled blood vessel 2120D that respectively correspond to the actual, imaged, blood vessels. As may be seen when comparing images 2101 and 2102, a shape, size, and position of each of the modeled blood vessels 2120 corresponds to a shape, size, and position of its corresponding blood vessels 2110 shown in retinal image 2101 however, the complexity of image 2102 showing a model of the corresponding blood vessels 2110 is greatly reduced.

Second retinal image 2103 shows a fifth blood vessel 2110E and a sixth blood vessel 2110F and image of a model of second retinal image 2104 shows a modeled fifth blood vessel 2120E and a sixth modeled blood vessel 2120F. As may be seen when comparing images 2103 and 2104, a shape, size, and position of each of the modeled blood vessels 2120 corresponds to a shape, size, and position of its corresponding blood vessels 2110 shown in retinal image 2103.

Third retinal image 2105 shows a seventh blood vessel 2110G, an eighth blood vessel 2101H, and a ninth blood vessel 2110I and image of a model of third retinal image 2106 shows a modeled seventh blood vessel 2120G, a modeled eighth blood vessel 2120H, and a modeled ninth blood vessel 2120I. As may be seen when comparing images 2105 and 2106, a shape, size, and position of each of the modeled blood vessels 2120 corresponds to a shape, size, and position of its corresponding blood vessels 2110 shown in retinal image 2105.

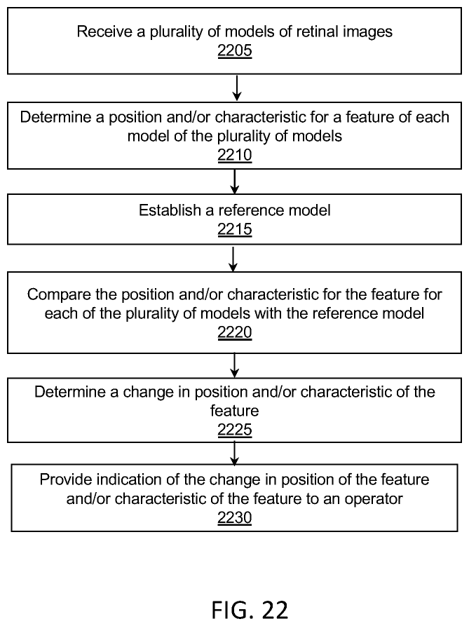

FIG. 22 provides a flowchart of an exemplary process 2200 for using predicted and/or modeled features of a retinal image, or a series of retinal images, to determine characteristics of the retina and/or track retinal/eye motion over time. Process 2200 may be executed by, for example, any of the systems and/or components disclosed herein using, for example, information and/or data received via any of the systems, devices, and/or components disclosed herein.

Initially, a plurality of models of retinal images (e.g., image of a model of first, second, and/or third retinal image 2102, 2104, and/or 2106, respectively) may be received, with each model corresponding to a different retinal image taken at a different point, or moment, in time. In some cases, the modeled retinal images may be part of a set corresponding to retinal images taken over a period of time (e.g., a 20 or 60 second video) and, in other cases, a time frame between when the various retinal images were captured may be much longer (e.g., weeks, months, or years).

In step 2210, a position (e.g., X- and/or Y-coordinates) and/or characteristic of a feature each model of the plurality of models may be determined. Exemplary characteristics include, but are not limited to, an orientation, position, width, length, size, and/or shape of the feature. Optionally, in step 2215, a reference model of the plurality of models may be established. In many cases, the reference model may correspond to the first-in-time retinal image upon which the models of the plurality of models are based.

In step 2220, the position and/or characteristic included in the reference model of step 2215 may be compared with a modeled retinal image received in step 2205 to determine differences therebetween (step 2225). In some cases, execution of step 2225 may include application of an algorithm and/or filter such as a Kalman and/or particle filter to track or otherwise determine motion of a modeled feature of a retinal image over time. Exemplary differences in characteristic that may be determined in step 2025 include, but are not limited to, changes (e.g., thickening, thinning, shortening, etc.) to features of the retina that may assist with the diagnosis, prognosis, and/or evaluation of a disease (e.g., multiple sclerosis, hypertension, etc.) state and/or progression. In step 2230, an indication of a change in position of a feature and/or a change in a characteristic of the model may be provided to an operator via, for example, a computer display device. In some instances, execution of process 2000 may enable faster and/or more efficient processing and/or analysis of a series of retinal images without sacrificing accuracy because when complex retinal images that include a plurality (10-100) different shades of grey are resolved into images that have binary (i.e., black and white) shading that distinguishes features of interest from background areas of the retina (e.g., areas covered with cells or photoreceptors), this enables more efficient identification of the features over a series of images and therefore faster and more efficient position tracking for the features of interest over a series of retinal images (e.g., a video).

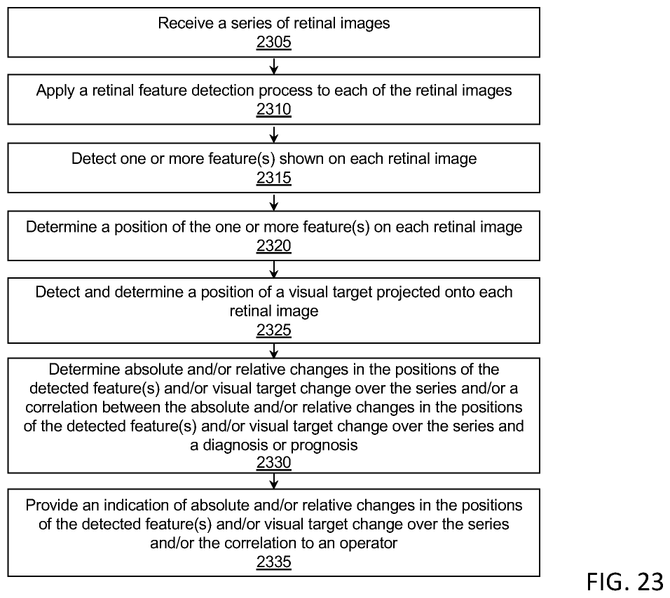

FIG. 23 provides a flowchart of an exemplary process 2300 for detecting and/or analyzing characteristics of retinal motion over time. Process 2300 may be executed by, for example, any of the systems and/or components disclosed herein using, for example, information and/or data received via any of the systems, devices, and/or components disclosed herein.

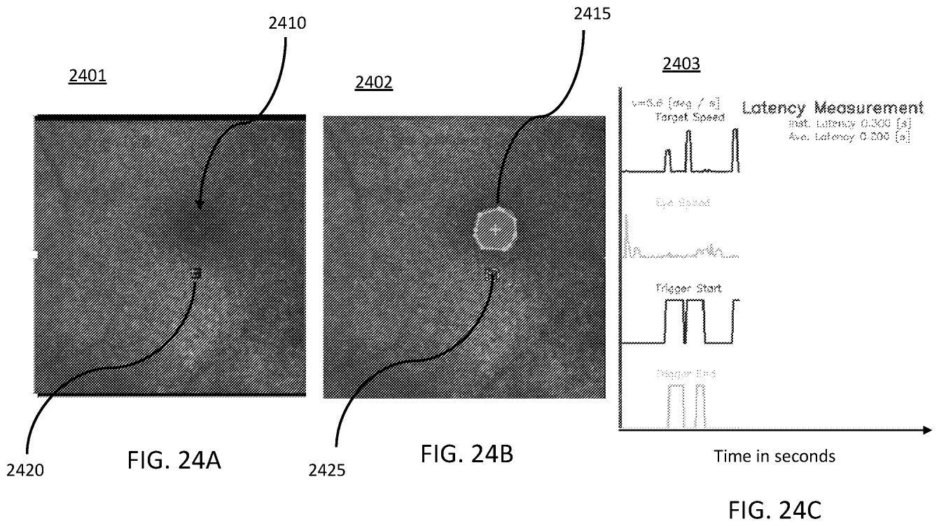

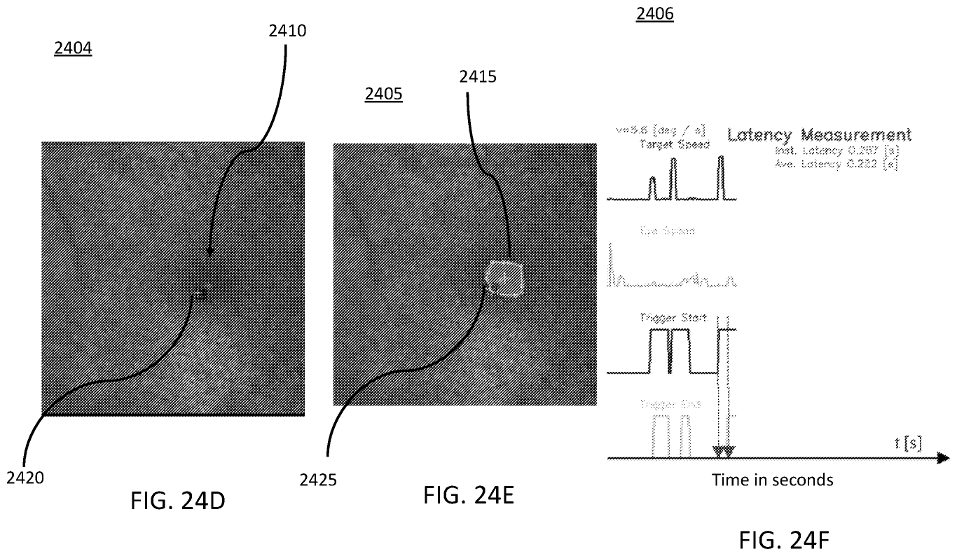

In step 2305, a series of retinal images, such as the retinal images described herein, may be received. FIGS. 24A and 24D provide exemplary retinal images 2401 and 2404 that may be received in step 2305 that are part of a set of 300 retinal image frames that are taken over a ten second interval time at a rate of 30 images per second, wherein retinal image 2401 corresponds to a frame 103 (taken at time 3.4 s) and retinal image 2404 corresponds to a retinal image of frame 113 (taken at time 3.8s). Retinal images 2401 and 2404 show the subject's fovea (a dark region of the image) 2410 and also a visual target (in the form of a tumbling “E”) 2420 projected upon the subject's retina at time=3.4s and time=3.8s, respectively.

In step 2310, a retinal feature detection process, such as the second version of the retinal feature detection model and/or algorithm generated via execution of process 2200 may be applied to each retinal image in the series to detect a feature of the retina shown or otherwise provided by the respective retinal images (step 2315). Exemplary features of the retina include, but are not limited to, fovea, macula, capillaries, capillary branches, vasculature, vascular branches, blemishes, injuries, and/or retinal photoreceptors. In step 2320, a position (e.g., X- and Y-coordinates) of the detected feature(s) shown on each of the retinal images included in the series may be determined. In step 2325, a visual target projected on to the retina (and visible in the retinal image) may be detected and a position (e.g., X- and Y-coordinates) of the visual target projected on to the retina within each of the images of the series may be determined.

FIGS. 24B and 24E provide modified retinal images 2402 and 2405, respectively, which are modified versions of retinal image 2401 and 2404, respectively. Modified retinal images 2402 and 2405 may be generated via, for example, execution of steps 2310-2325 wherein the fovea is the object detected in step 2310 and encircled with a foveal representation 2415 and a visual target 2425 in a first position is shown in FIG. 24B and visual target 2425 in a second position in FIG. 24E. Absolute and relative positions of foveal representation 2415 may be determined over time in order to, for example, determine a magnitude of foveal movement and/or speed (or velocity) of foveal movement. Visual target 2425 may be the same visual target (e.g., a tumbling E) projected onto the subject's retina over time and an absolute position of the visual target 2425 may be constant over time as it is always projected onto the retina from the same location in an optical array used to image the retina.

In step 2330, absolute and/or relative changes in the positions of the retinal feature(s) and/or visual target over the series of images may be determined and then provided to an operator and/or another system (step 2335). Exemplary operators include individuals who operate retinal scanning equipment as, for example, described herein. Optionally, execution of step 2330 may also include determining one or more correlations between the absolute and/or relative changes in the positions of the retinal feature(s) and/or visual target and one or more diagnosis or prognosis characteristics as may be stored in, for example, database 185. For example, changes in a caliber of a blood vessel of a retina over an interval of time (e.g., a 10-60 second video or 6 months or a year) may be interpreted to provide information regarding the subject's blood flow, rate of blood pulsation, and/or blood pressure which may be indicative of cardiovascular disease. Additionally, or alternatively, over time arteries can narrow, which may indicate the subject has hypertension. Additionally, or alternatively, a shape and/or size of retinal veins may develop a “beading” effect due to dysregulation of blood flow, which may indicate the patient has diabetes. Additionally, or alternatively newly appearing intraretinal vessels may indicate the subject is suffering from chronic diabetes. Thus, the ability to track blood flow may provide using the systems, devices, and/or methods disclosed herein may provide advantages over traditional methods to viewing blood flow rate and arterial and/or venal changes because they do not require use of injectable dyes like during traditionally performed fluorescein angiography.

In some cases, the systems, devices, and methods disclosed herein may be used to track disease progression, sometimes on a cellular level, over time. For example, retinal diseases like diabetic retinopathy, macular edema, vascular occlusions, and macular degeneration may cause alternation to the distribution of photoreceptors in the retina that can be identified and/or tracked over time on the individual cell level using the systems, devices, and/or methods disclosed herein. This greatly improves the ability to track disease progression with a finer level of granularity (e.g., on the cellular level) when compared with the traditionally used Optical Coherence Tomography (OCT) and/or Optical Coherence Tomography Angiography (OTCA), which can only identify larger magnitude changes to the retina and are incapable of detecting changes to the retina on the cellular level.

In another example, observations and/or analysis of fixational eye movement (e.g., the retinal motion that is being detected by the systems and devices disclosed herein) may indicate the presence and/or severity of one or more neurological conditions that may, for example, impact the pathophysiology of fixation and saccadic eye movements that may be detected via, for example, abnormalities in eye movements, which may be indicative of neurological disease. For example, with Huntington's disease, abnormalities in the basal ganglia region of the brain lead may to irregular microsaccades that may be detected using the systems, devices, and method disclosed herein and this may enable early detection of the disease and/or abnormalities in the pre-clinical/prodromal stage of the disease and/or may be used to track disease state and/or progression. Additionally, or alternatively, the systems, devices, and methods disclosed herein may be used to detect eye motion and/or features that are indicative of neurologic movement disorders such as Parkinson's Disease, multiple system atrophy, and progressive supranuclear palsy which may have similar clinical symptoms at disease onset will but have different patterns of abnormalities within fixational eye movements. In this way, the systems, devices, and methods disclosed herein may be helpful in differentiating between these (and other similar) conditions from one another and set and/or monitor a course of treatment.

In another example, the systems, devices, and methods disclosed herein may be used to diagnose and/or monitor ophthalmic disease (e.g., amblyopia and macular disease) and its impact fixational eye movement because fixation is directly related to visual quality/acuity because when an ophthalmic disease impacts visual acuity, the fixational eye movement pattern of the affected eye(s) will have patterns indicative of the ophthalmic disease and/or will change for a particular subject as their disease progresses.

The absolute and/or relative changes in the positions of the retinal feature(s) and/or visual target over time determined in step 2330 may be used to, for example, measure latency (i.e., how long it takes for a subject to move his or her eye to focus on the visual target), determine a magnitude of movement and/or speed (or velocity) of the features shown in the sequential retinal images, and by extension the subject's retinal movement, and/or determine whether the subject is able to move his or her eye to focus on the visual target (as may be indicated by, for example, whether the subject directs his or her fovea to a position that does not correctly align with the visual target (sometimes referred to as hypermetric or hypometric movements)). Additionally, or alternatively, the determinations of step 2330 may be used to measure and/or quantify, for example, fixational stability, drift, and/or microsaccadic movement of the subject's retina.

Continuing with the example of FIGS. 24A, 24B, 24D, 24E, and 24F, a change in absolute and/or relative position between visual target representation 2425 and foveal representation 2415 determined via execution of step 2330 may indicate whether (and how fast) the subject has moved his or her eye to look at the visual target 2425. For example, FIGS. 24A and 24B may represent when, in time, visual target 2425 is initially projected into the subject's retina and FIGS. 24D and 24E may be taken later in time and may be indicative of the subject moving his or her fovea to focus on the visual target. In some embodiments, absolute and/or relative movement between 24A/24B and 24D/24E may be observed and/or measured in order to, for example, determine how long it takes (also referred to herein as “latency”) for the subject to move his or her fovea 2410 and/or foveal representation 2415 to focus on visual target 2420 and/or visual target representation 2425. Indications of relative and/or absolute changes in movement of the retinal feature of the foveal representation 2415 and visual target representation 2425 may be provided to the user as a series of one or more measurements and/or graphs as shown in analysis results window 24C and 24F, which provide analysis results for FIGS. 24B and 24E, respectively. For example, FIGS. 24C and 24F provide measurements for instantaneous latency times (0.300s for FIG. 24C; 0.237s for FIG. 24F) and average latency (0.200s for FIG. 24C; 0.222s for FIG. 24F) as well as a graphs (all as a function of time) of visual target speed (first graph), eye speed (second graph), when the trigger (i.e., projection of visual target 2420 on the retinal) starts (third graph), when the trigger (i.e., projection of visual target 2420 on the retinal) ends (fourth graph).

1. A system, comprising a scanning laser ophthalmoscope (SLO) comprising:
monocular imaging optics configured to image a retina of a right or left eye of a subject, the monocular imaging optics comprising:
a scanning radiation source (870) arranged and configured to emit a beam of scanning radiation for imaging the subject's retina toward a fiber collimator (872);
a fiber collimator (872) arranged and configured to receive the beam of scanning radiation from the scanning radiation source (870), collimate the beam of scanning radiation, thereby generating a collimated beam of scanning radiation, and direct the collimated beam of scanning radiation to a first beam splitter (865);
the first beam splitter (865) arranged and configured to direct the collimated beam of scanning radiation to a scan path defocus correction assembly (824);
the scan path defocus correction assembly (824) arranged and configured to receive the collimated beam of scanning radiation, apply a defocus or spherical equivalent correction of a subject's eye or eyes to the collimated beam of scanning radiation, thereby generating a corrected beam of scanning radiation, and direct the corrected beam of scanning radiation through an iris (897) toward a first mirror (845);
the first mirror (845) arranged and configured to direct the corrected beam of scanning radiation to a fast-scanner optical element (867);
the fast-scanner optical element (867) arranged and configured to receive the corrected beam of scanning radiation and direct the corrected beam of scanning radiation toward a slow-scanning optical element (866);
the slow-scanning optical element (866) arranged and configured to receive the corrected beam of scanning radiation and direct the corrected beam of scanning radiation toward an optical element (850);
the optical element (850) arranged and configured to receive the corrected beam of scanning radiation and direct the corrected beam of scanning radiation to a second beam splitter (830);
the second beam splitter (830) arranged and configured to direct the corrected beam of scanning radiation toward a relay element (835B); and
the relay element (835B) arranged and configured direct the corrected beam of scanning radiation onto a subject's pupil.

2. The system of claim 1, wherein the scan path defocus correction assembly (895) is opto-mechanically controlled.

3. The system of claim 1, wherein the scan path defocus correction assembly (895) comprises two lenses.

4. The system of claim 1, wherein the defocus or spherical equivalent correction of a subject's eye or eyes applied to the collimated beam of scanning radiation by the scan path defocus correction assembly (895) is within a range of −12 diopters to +12 diopters.

5. The system of claim 1, wherein the defocus or spherical equivalent correction applied to the collimated beam of scanning radiation by the scan path defocus correction assembly (895) is responsive to an analysis of retinal image quality.

6. (canceled)

7. The system of claim 1, wherein the fast-scanning optical element (867) is arranged and configured to direct the corrected beam of scanning radiation toward the slow-scanning optical element along a first scanning dimension.

8. The system of claim 1, wherein the slow-scanning optical element (866) is arranged and configured to direct the corrected beam of scanning radiation toward the optical element (850) along a second scanning dimension.

9. The system of claim 1, further comprising an acousto-optic modulator (AOM) arranged and configured to generate a fixation target for viewing by the subject.

10. The system of claim 1, wherein a camera (888) is used in aiding alignment of the subject with the imaging optics.

11. The system of claim 1, further comprising a detector assembly, the detector assembly comprising:
a focusing lens (875) arranged and configured to receive scanning radiation reflected from the subject's retina via the first beam splitter (865) and focus the radiation reflected from the subject's retina onto an imaging system (880); and
the imaging system (880) arranged and configured to receive scanning radiation reflected from the subject's retina from the first beam splitter (865) and communicate an indication of the scanning radiation reflected from the subject's retina to an external computing device.

12-18. (canceled)

19. A method comprising:
receiving, by a processor, a set of detection path signals from an optical array, the detection path signals corresponding to a plurality of scans of a subject's retina taken over a time interval;
processing, by the processor, the set of detection path signals to generate a plurality of images of the subject's retina;
determining, by the processor, a signal to noise ratio for each retinal image of the plurality of images of the subject's retina;
determining, by the processor, whether the signal to noise ratio for each retinal image is below a threshold value and, if so, removing any set retinal image with a signal to noise ratio below the threshold from the plurality of images of the subject's retina, thereby generating an edited set of images of the subject's retina.

20-22. (canceled)

23. The method of claim 19, further comprising:
receiving, by the processor, a preferred luminance level range for retinal images;
determining, by the processor, whether a luminance level for each retinal image of the edited set of images of the subject's retina falls within the preferred luminance level range and, if not, adjusting the luminance level for each of the retinal images included in the edited set of images of the subject's retina that does not fall within the preferred luminance level range for retinal images.

24. The method of claim 19, further comprising:
analyzing, by the processor, each retinal image included in the edited set of images of the subject's retina to determine differences therebetween; and
providing, by the processor, an indication of a determined difference to an operator.

25. The method of claim 19, further comprising:
analyzing, by the processor, each retinal image included in the edited set of images of the subject's retina to determine a characteristic thereof; and
comparing, by the processor, a determined characteristic of at least two retinal images to one another;
providing, by the processor, an indication of the comparison to an operator.

26. The method of claim 25, wherein the determined characteristic is a position of a feature shown in the at least two retinal images, the method further comprising:
determining, by the processor, a velocity of retinal motion using the position of the feature shown in the at least two retinal images and a time interval between the capturing of the detection path data used to generate the at least two retinal images.

27. The method of claim 25, wherein the determined characteristic is a direction of retinal motion, a magnitude of retinal motion, a speed of retinal motion, a magnitude of drift, and a velocity of drift.

28. The method of claim 19, wherein determining the signal to noise ratio includes performing a frequency spectrum analysis on each retinal image.

29. The method of claim 28, wherein determining the signal to noise ratio includes determining a relationship between frequency and intensity for each retinal image of the plurality of images of the subject's retina.

30-38. (canceled)
